(19) United States
(12) Patent Application Publication
(10) Pub. No.: US 2023/0193409 A1
(43) Pub. Date: Jun. 22, 2023

(54) PHAGE-ENCODED AcrVIA1 FOR USE AS AN INHIBITOR OF THE RNA-TARGETING CRISPR-Cas13 SYSTEMS

(71) Applicant: THE ROCKEFELLER UNIVERSITY, New York, NY (US)

(72) Inventors: Alexander Meeske, New York, NY (US); Luciano Marraffini, New York, NY (US)

(21) Appl. No.: 17/995,401

(22) PCT Filed: Mar. 30, 2021
(86) PCT No.: PCT/US21/24979

§ 371 Date: Oct. 3, 2022

Related U.S. Application Data
(60) Provisional application No. 63/004,940, filed on Apr. 3, 2020.

(51) Int. Cl. C12Q 1/70; C12N 15/10

(52) U.S. Cl. CPC C12Q 1/701; C12N 15/1055; C12N 15/1048; C12Q 1/6816

(57) ABSTRACT

Provided is an anti-CRISPR protein (AcrVIA1), which acts as an inhibitor of the nuclease of Cas13. Cas13 recognizes complementary viral transcripts to trigger the degradation of both host and viral RNA during the type VI CRISPR-Cas antiviral response. AcrVIA1 is provided as an isolated or recombinantly expressed protein comprising the sequence of SEQ ID NO:1, or derivatives thereof, expression vectors that encode the same sequence, and methods of making and using proteins that comprise the same sequence, or derivatives thereof, for inhibiting the function of Cas13 and/or protein complexes and/or ribonucleoprotein complexes that comprise Cas13. The disclosure further includes use of the described inhibitor protein in improved diagnostic assays that include Cas13. Inclusion of the inhibitor is expected to preclude a requirement to reverse transcribe and/or create cDNA amplifications of the particular RNA that is the subject of the analysis.

### CROSS-REFERENCE TO RELATED APPLICATIONS

This application claims priority to U.S. Provisional patent application No. 63/004,940, filed on Apr. 3, 2020, the disclosure of which is incorporated herein by reference.

### STATEMENT REGARDING FEDERALLY SPONSORED RESEARCH OR DEVELOPMENT

This invention was made with government support under grant no. 1DP1GM128184-01 awarded by the National Institutes of Health. The government has certain rights in the invention.

### FIELD

The present disclosure relates generally to CRISPR inhibition, and more specifically to proteins and derivatives thereof for use in inhibiting Cas13.

### BACKGROUND

Clustered, regularly interspaced, short palindromic repeats (CRISPR) systems and CRISPR-associated (Cas) proteins are prokaryotic adaptive immune systems that protect their hosts from invasion by viruses (1) and plasmids (2). CRISPR loci contain short DNA repeats separated by spacer sequences of foreign origin (3-5). To achieve immunity, the locus is transcribed and processed into small CRISPR RNAs (crRNAs), which associate with RNA-guided Cas nucleases (6) to locate and cleave complementary nucleic acid sequences (protospacers) (7-11). CRISPR systems are categorized into six types (I-VI) and 33 subtypes, which differ in their cas gene content and mechanism of immunity (12). While most types neutralize invaders through destruction of their DNA, Cas13, the RNA-guided nuclease of type VI systems, unleashes non-specific RNA degradation (trans-RNase activity) upon recognition of a phage target transcript (7, 13, 14). The cleavage of host transcripts leads to a growth arrest that prevents further propagation of the phage, allowing the uninfected cells in the population to survive and proliferate (14). Because the phage genome is not directly affected by Cas13, it continues to produce target transcripts, leading to a persistent activation of the nuclease and growth arrest (14).

Presumably in response to the pressure imposed by CRISPR-Cas immunity, phages evolved anti-CRISPR (Acr) proteins, small proteins (usually<150 aa) that are produced during infection and inactivate Cas nucleases (15). Acrs also exhibit exceptional diversity of sequences and mechanisms and, with few exceptions, specifically inhibit one CRISPR subtype. About 50 families of Acrs have been discovered that inhibit types I, II, III or V CRISPR-Cas systems (15-20). A characteristic of type I and II Acrs is that they rely on multiple rounds of phage infection to completely inactivate their cognate CRISPR system (21, 22). Expression of the phage-encoded Acr occurs shortly after infection, but it is not sufficient to neutralize all the active Cas nucleases inside the cell. Instead, only a fraction of the nucleases is inhibited per infective cycle, resulting in host immunosuppression rather than a complete halt of the CRISPR-Cas immune response (21, 22). As a consequence, the success of the Acr is highly dependent on the strength of the CRISPR-Cas immune response and on the multiplicity of infection (MOI): the presence of Cas nucleases programmed to target multiple sites in the viral genome, or a low concentration of phage prevent Acrl and AcrII inhibitors from overcoming immunity. But it is believed that no Acrs have been previously reported to inhibit type VI CRISPR-Cas immunity. Thus, there is an ongoing and unmet need to identify Acrs that have this function so that they can be adapted for use in deliberate inhibition of the pertinent cas enzymes, and for other proposes. The present disclosure is pertinent to these needs.

### SUMMARY OF THE DISCLOSURE

The crRNA-guided nuclease Cas13 recognizes complementary viral transcripts to trigger the degradation of both host and viral RNA during the type VI CRISPR-Cas antiviral response. Whether and how viruses can counteract this immunity is not known. We describe a listeriophage (ΦLS46) encoding an anti-CRISPR protein (AcrVIA1) that inactivates the type VI-A CRISPR system of Listeria seeligeri. Using genetics and biochemistry we demonstrate that AcrVIA1 interacts with the guide-exposed face of Cas13a to prevent access to the target RNA and the conformational changes required for nuclease activation. Unlike inhibitors of DNA-cleaving Cas nucleases, which cause limited immunosuppression and require multiple infections to bypass CRISPR defenses, a single dose of AcrVIA1 delivered by an individual virion can completely dismantle type VI-A CRISPR-mediated immunity.

ArcfVIA1 has the following amino acid sequence:

(SEQ ID NO: 1)
MIYYIKDLKVKGKIFENLMNKEAVEGLITFLKKAEFEIYSRENYSKYNK
WFEMWKSPTSSLVFWKNYSFRCHLLFVIEKDGECLGIPASVFESVLQIY
LADPFAPDTKELFVEVCNLYECLADVTVVEHFEAEESAWHKLTHNETEV
SKRVYSKDDDELLKYIPEFLDTIATNKKSQKYNQIQGKIQEINKEIATL
YESSEDYIFTEYVSNLYRESAKLEQHSKQILKEELN.

The disclosure provides compositions comprising proteins comprising this sequence, or derivatives thereof, fusion proteins comprising this sequence, or derivatives thereof, expression vectors that encode this sequence, and methods of making and using proteins that comprise this sequence, or derivatives thereof, for inhibiting the function of Cas13 and/or protein complexes and/or ribonucleoprotein complexes that comprise Cas13. The disclosure further includes use of the described inhibitor protein in diagnostic assays that include Cas13. Inclusion of the inhibitor is expected to provide certain improvements in diagnostic tests where samples containing or suspected of containing RNA signatures are evaluated, and may preclude a requirement to reverse transcribe and/or create cDNA amplifications of the particular RNA that is the subject of the analysis.

### BRIEF DESCRIPTION OF THE FIGURES

FIG. 1. AcrVIA1 inhibits type VI-A CRISPR-Cas immunity against plasmids and phages. (A) Transfer of a conjugative plasmid with or without the spc4 target of the L. seeligeri SLCC3954 type VI-A CRISPR-Cas system into different strains: wild-type (WT), Δspc Δcas13a or WT harboring the ΦLS46 or ΦLS46 ΔacrVIA1 prophages. Ten-fold dilutions of transconjugants were plated on selective media. (B) Schematic of the ΦLS46 genome showing the four main transcription units (acr in blue; lysogeny cassette in red; early- and late-expressed lytic genes in green and purple, respectively). gp2 was renamed acrVIA1. The locations of the targets of spacers used in this disclosure are shown in grey. (C) Same as (A) but using strains carrying plasmids to express different acr genes from ΦLS46. (D) Detection of phage propagation after spotting ten-fold dilutions of WT, A 1-4 or AacrVIA1 phage ΦLS46, on lawns of L. seeligeri ΔRM Δspc or ΔRM ΩspcE2. (E) Same as (D) but spotting ΦLS59 into lawns of L. seeligeri ΔRM Δspc, ΔRM Ωspc59 or ΔRM Ωspc59/pgp2. (F) Growth of WT, Δspc and WT/pgp2 L. seeligeri strains expressing a spc4 target RNA under the control of an anhydrotetracycline-inducible promoter, measured as OD600 over time after addition of the inducer.

FIG. 2. AcrVIA1 interacts with Cas13a^{cRNA} to prevent binding of the target RNA and RNase activation. (A) cis-RNA cleavage time course with purified L. seeligeri Cas13a-His6, AcrVIA1 and/or AcrVIA1-3xFLAG using radiolabeled non-target or spc2-target RNA substrates. The products of degradation after 5, 10, and 20 minutes were analyzed by PAGE. (B) trans-RNA cleavage time course as in (A) but using a radiolabeled non-target RNA substrate in the presence of unlabeled non-target or spc2-target RNA. (C) Anti-FLAG immunoprecipitation using protein extracts from L. seeligeri cells expressing either Cas13a-His6 alone or co-expressing AcrVIA1-3xFLAG. The His6 and FLAG epitopes, as well as the σ^{A} protein were detected via western blot. (D) EMSA of radiolabeled non-target or spc2-target RNAs in the presence of dCas13a-His6, with or without AcrVIA1-3xFLAG.

FIG. 3. Site-directed mutagenesis of AcrVIA1. (A) Transfer of conjugative plasmid with or without spc4 target of the L. seeligeri type VI CRISPR-Cas system into WT L. seeligeri harboring plasmid-borne wild-type or mutant alleles of acrVIA1-3xflag. (B) Anti-Flag immunoblot of AcrVIA1 mutants tested in (A), and anti-σ^{A} loading control.

FIG. 4. AcrVIA1 enables full phage escape from type VIA CRISPR-Cas immunity. (A) Efficiency of plaquing (relative to the number of plaques formed in lawns of L. seeligeri ΔRM Δspc) of phages ΦLS46 or ΦLS46 AacrVIA1 in lawns of bacteria expressing spcA1, spcE1, spcE2 or all three (3 spc). Error bars represent SEM from 3 biological replicates. (B-F) Growth of L. seeligeri ΔRM Δspc (B), ΔRM ΩspcE1 (C), ΔRM ΩspcE2 (D), ΔRM ΩspcA1 (E) and ΔRM Ωspc (F), measured as OD₆₀₀ over time, infected with ΦLS46 or ΦLS46 ΔacrVIA1 phages, or uninfected. The average curves of three different replicates are reported, with +/−SEM values shown in lighter colors.

FIG. 5. Acr screen in listeriophages. (A) Diagram of L. seeligeri SLCC3954 genetic elements modified according to this disclosure. (B) Detection of phages in lysates of L. seeligeri (11 phages isolated) or L. monocytogenes (4 phages isolated) strains after treatment with mitomycin C. Ten-fold dilutions of lysate were spotted on lawns of L. seeligeri ΔRM Δspc. (C) Confirmation of lysogen formation by the phages isolated in (B). Putative lysogens were treated with mitomycin C to induce and detect integrated prophages. Ten-fold dilutions of induced culture filtrates from each lysogen were spotted on lawns of L. seeligeri ΔRM Δspc. For ΦLS6 and ΦLS48, lysogens were less stable and spontaneous plaques were detectable during growth of the uninduced lysogen. (D) Transfer of a conjugative plasmid with or without the spc4 target of the L. seeligeri SLCC3954 type VI-A CRISPR-Cas system into different strains: wild-type (WT), Δspc Δcas13a or WT harboring the ΦLS46, ΦLS57, Φ10403S, ΦLS4, ΦLS48, ΦLS6, U153, or ΦEGDe prophages. Ten-fold dilutions of transconjugants were plated on selective media.

FIG. 6. AcrVIA1 inhibits type VI-A CRISPR-Cas targeting of plasmids and phages. (A) Transfer of a conjugative plasmid with or without the spc2 or spc4 target of the L. seeligeri SLCC3954 type VI-A CRISPR-Cas system into strains ΔRM Δspc, ΔRM Ωspc2 or ΔRM Ωspc4. Ten-fold dilutions of transconjugants were plated on selective media. (B) Detection of phage propagation after spotting ten-fold dilutions of the phages ΦLS46 or ΦLS59, on lawns of L. seeligeri ΔRM Δspc or ΔRM Ωspc59. (C) Schematic of the ΦLS46 genome showing the four main transcription units (acr in blue; lysogeny cassette in red; early- and late-expressed lytic genes in green and purple, respectively). The location of the targets of the spacers used in this disclosure are shown in grey. Top and bottom locations refer to the DNA strand that is transcribed to produce a target transcript that is complementary to the crRNA derived from each spacer. (D) Same as (B) but spotting phages ΦLS46 or ΦLS46 ΔacrVIA1 on lawns of bacteria expressing crRNAs from the spacers shown in (C).

FIG. 7. Purification and functional test of Cas13a-His6 and AcrVIA1-3xFLAG. (A) SDS-PAGE of Cas13a-His6 after expression and purification from L. seeligeri. M, protein size marker; E, elution. (B) Same as (A) but for AcrVIA1 purified from E. coli. (C) Transfer of a conjugative plasmid with or without the spc4 target of the L. seeligeri SLCC3954 type VI-A CRISPR-Cas system into L. seeligeri strains expressing Cas13a-His6 and harboring either an empty vector or p(AcrVIA1-3xFLAG). Three ten-fold dilutions of transconjugants were plated on selective media.

FIG. 8. AcrVIA1 enables full phage escape from type VI-A CRISPR-Cas immunity. (A) Efficiency of plaquing (relative to the number of plaques formed in lawns of L. seeligeri (ΔRM Δspc)) of phages ΦLS46 or ΦLS46 ΔacrVIA1 in lawns of bacteria expressing spcL1, spcL2, spcL4, spcL5, spcL6, spcL7 or spcL8. These spacers target transcripts produced by the late-expressed region of ΦLS46; shown in FIG. 6C (B-H) Growth of L. seeligeri (ΔRM ΩspcL1), (ΔRM ΩspcL2), (ΔRM ΩspcL4), (ΔRM ΩspcL5), (ΔRM ΩspcL6), (ΔRM ΩspcL7) and (ΔRM ΩspcL8), measured as OD600 over time, infected with ΦLS46 or ΦLS46 ΔacrVIA1 phages, or uninfected. The average curves of three different replicates are reported, with +/−SEM values shown in lighter colors.

FIG. 9. AcrVIA1 does not inhibit Leptotrichia buccalis Cas13a. cis-RNA cleavage time course with purified L. buccalis Cas13a, AcrVIA1 and/or AcrVIA1-3xFLAG using radiolabeled spc2-target RNA substrates. Cas13a was present at 10 nM, synthetic crRNA at 10 nM, and AcrVIA1 or AcrVIA1-3xFLAG was added at 400, 100, 10, or 1 nM. The products of degradation after 5, 10, and 20 minutes were analyzed by denaturing PAGE.

FIG. 10. RNA-seq during ΦLS46 infection. Strand-specific read coverage of phage-mapped reads is plotted along the ΦLS46 genome, and normalized to total reads in each sample. Targeting location of the spacers is also shown.

FIG. 11. Data demonstrating enhanced Cas13 nuclease activity on non-targeted RNA in the presence of AcrVIA1. Cas13 nuclease activity on non-targeted RNA is dependent on the order of addition of the inhibitor with respect to interaction of Cas13 with guide-targeted RNA. As described further below, if AcrVIA1 is added to the reaction containing Cas13 before adding the target RNA, then AcrVIA1 prevents target RNA binding and no Cas13 activity is observed (“Acr first” lane). In contrast, if AcrVIA1 is added to the reaction shortly after Cas13, the target RNA and a guide RNA targeted to the targeting RNA are combined, the AcrVIA1 prolongs the non-specific RNA nuclease activity.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein. The disclosure includes all polynucleotide and amino acid sequences described herein, and all DNA and RNA sequences that encode any polypeptide as described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included. Sequences of from 80-99.99% identical to any sequence (amino acids and nucleotide sequences) of this disclosure are included. If reference to an amino acid or nucleotide sequence is made to by way of a database entry, the sequence corresponding to that database entry as it exists on the effective filing date of this application or patent is incorporated herein by reference.

In embodiments, a protein of this disclosure comprises SEQ ID NO:1, or a modified version thereof, wherein the modified version comprises a truncated protein, a fusion protein, or mutated version of said protein.

In embodiments, the disclosure provides compositions and methods for use in, for example, type VI CRISPR-Cas13 anti-CRISPR applications. As such, the proteins of this disclosure may be used to control Cas13 RNA editing activity. The disclosure therefore provides a means for controlling Cas13 activity in a variety of settings, including but necessarily limited to therapeutic, veterinary and agricultural, and research-based implementations. The proteins of this disclosure, and compositions comprising them, may be used in any cell type, including but not limited to prokaryotes and eukaryotes. In embodiments, a system comprising a Cas13 protein of this disclosure is used to modulate RNA editing in any of bacteria, archaea, plants, and animal cells, that latter of which include but are not necessarily limited to cells of insects, fish, avian animals, and mammals, including but not limited to humans. The proteins of this disclosure may also be used to modulate the activity of viruses, including but not limited to any virus having an RNA genome, whether single or double stranded, or a single strand or segmented genome, or any virus that uses an RNA intermediate, and any virus, such as virus with a DNA genome that is used to produce an RNA transcript. Further, the inhibition may be pertinent to Cas13 editing of any type of RNA, including but not necessarily limited to mRNA, hnRNA, miRNA, snoRNA, RNA produced within organelles, and the like. Inhibition of RNA editing by Cas13 may be performed in vitro or in vivo. Ex vivo modification of cells to express a protein of this disclosure for use in subsequent therapeutic or other approaches is also encompassed by this disclosure.

In embodiments, the disclosure provides for using a protein of this disclosure to inhibit, for example, crRNA from properly complexing with Cas13a, and/or inhibits binding of Cas13 or a complex comprising Cas13 to a complementary target RNA. In embodiments, the disclosure provides for inhibition of conformational changes required for the activation of the RNase function of Cas13a.

In embodiments, the disclosure provides a contiguous segment of the amino acids of SEQ ID NO:1 that is sufficient to partially or fully inhibit the RNA cleavage function of Cas13a, such as by preventing association of the nuclease with a targeted protospacer RNA. Thus, in embodiments, the disclosure provides a polypeptide comprising a contiguous segment of SEQ ID NO:1 that comprises from 10-232 contiguous amino acids of SEQ ID NO:1, including all integers and ranges of integers there between, or a contiguous polypeptide that is at least 80% identical to such a segment of SEQ ID NO:1. In embodiments, a contiguous segment of a protein of this disclosure consists of SEQ ID NO:1. In embodiments, a protein comprising a contiguous sequence that consists of SEQ ID NO:1 is functional relative to a shorter, or a mutated version of SEQ ID NO:1.

In embodiments, pharmaceutical compositions comprising an inhibitor protein of this disclosure are provided. In embodiments, a pharmaceutical composition comprises at least one pharmaceutically acceptable additive.

In embodiments, the disclosure provides an expression vector encoding the described protein. In embodiments, a sequence encoding the described inhibitor protein is operably linked to an inducible promoter so that expression of the inhibitor protein can be controlled, such as to inducibly express the protein in order to inhibit or completely stop Cas13-based RNA editing.

In embodiments, the disclosure provides for administering to cells, tissues, or an organism, or a combination thereof, an inhibitor protein of this disclosure, or a polynucleotide encoding the inhibitor protein. In embodiments, an effective amount of the inhibitor protein that is sufficient to inhibit or stop Cas13-based RNA editing is introduced into cells, tissue, or an organism. In embodiments, and without intending to be limited by any particular theory, it is considered, at least for use in bacteria, that a single copy of the gene encoding the described inhibitor protein will be sufficient to stop Cas13a RNA degradation in a single bacterium. In embodiments, an amount of the described inhibitor protein that is administered and is sufficient to inhibit or stop Cas13-based RNA editing is less than the amount of Cas-enzyme inhibition determined from any suitable reference, e.g., the amount of inhibitor protein is less than a control value. Suitable control values can be obtained from other proteins, which may include known protein inhibitors of other Cas-enzymes, including but not necessarily limited to Cas9 enzyme protein-based inhibitors. Thus, it is considered that the presently provided proteins are more potent than previously described Cas-enzyme inhibitors, insofar as their capacity to inhibit Cas13a nuclease activity. Accordingly, in embodiments, the disclosure comprises introducing or causing the expression of an inhibitor protein described herein such that the inhibitor protein functions to inhibit or stop RNA editing within a cell that also comprises a Cas13-based RNA editing system, which may comprise an engineered system that is designed to specifically target any particular RNA, or target more than one RNA. As such, the system comprises at least a Cas13 protein, and a guide RNA targeted to a target RNA. In embodiments, the Cas13 activity that is inhibited using a described inhibitor protein functions comprises an L. seeligeri type VI-A Cas13a protein. The sequence of this VI-A Cas13a protein is known in the art and is available from, for example, GenBank accession number WP_012985477.1, the sequence from which is incorporated herein as of the effective filing date of this application or patent.

In embodiments, the disclosure provides for editing RNA in one or more cells using a Cas13 protein, such as the L. seeligeri type VI-A Cas13a, protein, as a component of a Cas13-CRISPR RNA editing system. The RNA editing system comprises the Cas13a protein or a vector encoding it, and may further comprise one or more crRNAs and/or guide RNAs, or one or more vectors encoding crRNAs and/or guide RNAs so that respective RNA is expressed in the cell. Additional CRISPR proteins may be included, such as any additional protein that is required for Cas13a RNA editing to function. In general, the guide RNA is designed to target a protospacer present in a targeted RNA. The protospacer is not particularly limited. In embodiments, Cas13a targeting efficiency decreases substantially if the 3′ end of the target RNA is flanked by nucleotides homologous to a CRISPR repeat sequence, such as a sequence comprising GTTTAGT (SEQ ID NO:2), and thus suitable modifications of the target RNA can be taken into account when implementing aspects of the disclosure.

In embodiment, the method comprises allowing RNA editing catalyzed at least in part by the Cas13a protein, and at a desired time, causing the RNA editing to be inhibited or stopped by introducing into the cell an inhibitor protein of this disclosure, such as by introducing the protein into the cell directly, or using a delivery system, or by inducing its expression from a controllable promoter. In embodiments, nuclease activity of the Cas13a is suppressed in the cells of an organism wherein an adverse result is experienced by the individual as a consequence of the Cas13a RNA editing. In embodiments, the individual experiencing the adverse event is being treated for viral infection. In embodiments, the individual is being treated with a Cas13 used as an anti-viral therapeutic against an infection by an RNA virus. In embodiments, the individual is infected with a coronavirus.

In embodiments, a Cas13a CRISPR editing system, and/or a protein of this disclosure, is administered to bacteria using a modified bacteriophage, or by packaged phagemids. In embodiments, a Cas13a CRISPR editing system, and/or an inhibitor protein of this disclosure, is encoded by a conjugative plasmid. In embodiments, providing a conjugative plasmid encoding an inhibitor protein of this disclosure may cause the inhibitor protein to be expressed in other bacteria by horizontal plasmid transfer. In embodiments, Cas13a system and a means for controllable inhibitor protein expression may be introduced into bacteria (or eukaryotic cells) that are used for industrial purposes, such as in the food or beverage industry, or for the production of biological agents. In embodiments, bacteria that are modified as described herein comprise lactic acid bacteria. In additional and non-limiting embodiments, the Cas13a and a means for controllable inhibitor protein expression are introduced into pathogenic bacteria, including but not limited to Enterococcus faecium, Staphylococcus aureus, Klebsiella pneumoniae, Acinetobacter baumanii, Pseudomonas aeruginosa, or Enterobacter spp. In embodiments, the system is introduced into biofuel producing bacteria, such as Zymomonas mobilis. In embodiments, the system is introduced into plant-associated bacteria, such as Agrobacterium tumiefaciens. Thus, in embodiments, the disclosure includes expression of the described inhibitor protein in a heterologous host. A heterologous host means any cell that does not comprise a polynucleotide that encodes the described inhibitor protein prior to being modified to express the described inhibitor protein as described herein.

In embodiments, the targeted RNA that is edited by the Cas13a system in the absence of a described inhibitor protein encodes and is translated into any protein of interest, which may include but is not limited to a selectable or detectable marker. In embodiments, the targeted RNA encodes a protein that produces a detectable signal, thereby permitting analysis of targeting using a system of this disclosure by detecting an absence or a reduction in the detectable signal when the inhibitor protein is present and functional within the cell. In embodiments, the detectable signal is produced by a fluorescent protein. In embodiments, the targeted RNA encodes an antibiotic resistance protein, or a virulence factor. Thus, in embodiments, a change in antibiotic resistance or virulence can be determined by operation of a functional inhibitor protein. In embodiments, an inhibitor protein of this disclosure is used during analysis of any of RNA editing, knock-down and/or RNA visualization applications.

For use in eukaryotic cells, the described inhibitor protein can be modified to enhance its utility, such as by including a nuclear localization signal as a component of the inhibitor protein. Thus the disclosure includes use of at least one nuclear localization signal (NLS) in the described inhibitor protein. In general, a suitable NLS includes one or more short sequences of positively charged lysines or arginines exposed on the protein surface. Further, the described inhibitor proteins may also be modified by including, for example, a suitable purification tag, such as a poly-histidine tag.

In embodiments, a system of this disclosure is introduced into eukaryotic cells using, for example, one or more expression vectors, or by direct introduction of ribonucleoproteins (RNPs). In embodiments, expression vectors comprise viral vectors. In an embodiment, adenoviral vectors may be used, and many such vectors are known in the art and can be adapted for use with eukaryotic cells when provided the benefit of this disclosure.

The following description and examples are intended to illustrate embodiments of, but not limit the disclosure.

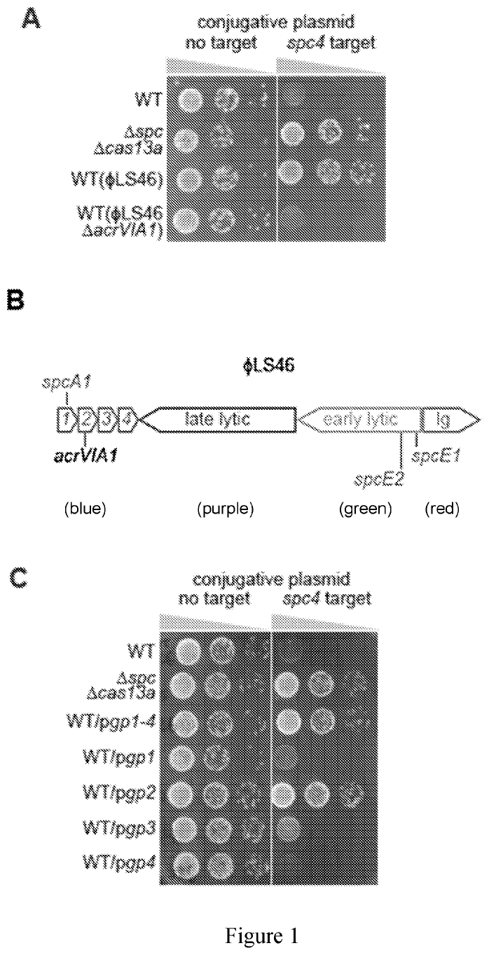

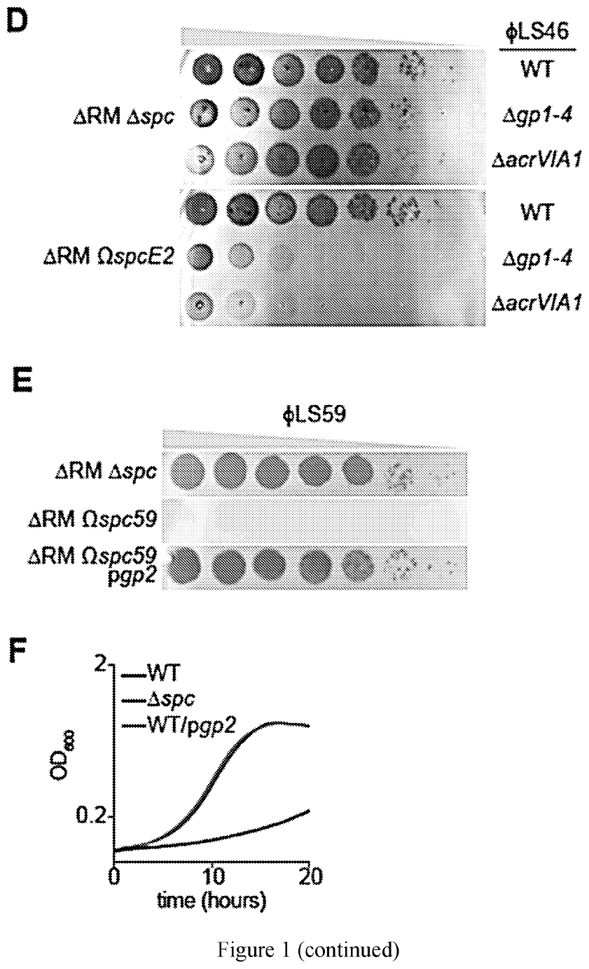

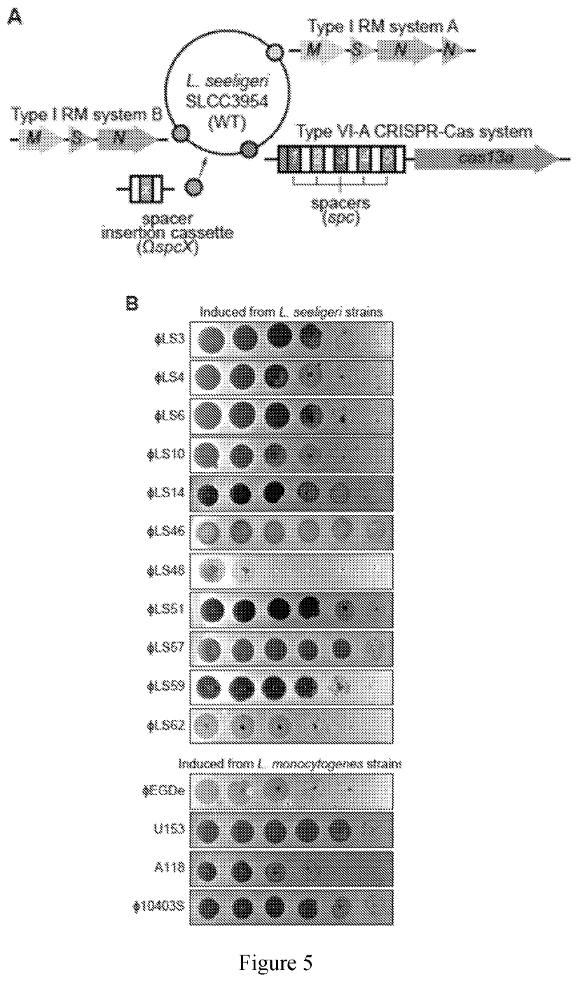

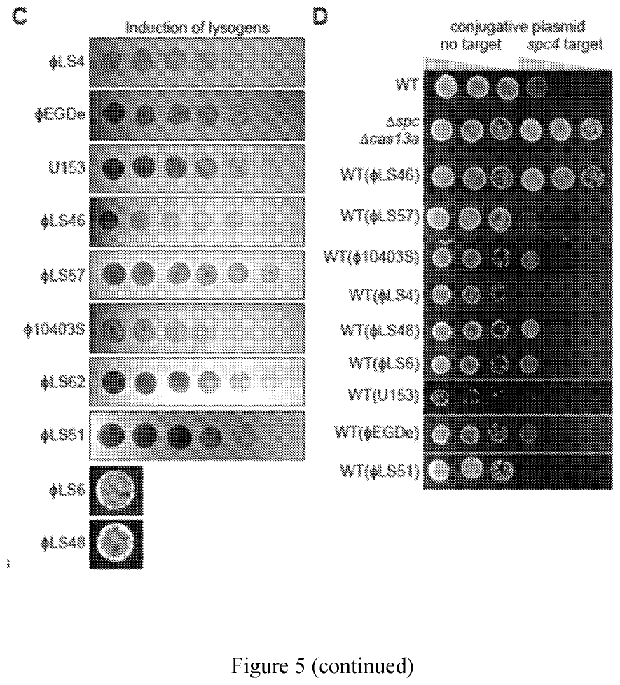

In arriving at aspects of the present disclosure, we first obtained temperate phages from a collection of 62 environmental isolates of Listeria spp., a natural host for type VI-A CRISPR-Cas systems. We treated cultures with mitomycin C and looked for the ability of the supernatants to form plaques on an agar lawn seeded with a mutant of Listeria seeligeri SLCC3954 (23) lacking its two restriction-modification systems and the type VI-A CRISPR array (L. seeligeri ΔRM Δspc), FIG. 5A). We isolated 15 phages that formed plaques (FIG. 5B), which we used to infect wild type L. seeligeri SLCC3954 (WT) and obtain 10 lysogens carrying different prophages in their genomes (FIG. 5C). We then tested each lysogen for Cas13a-mediated immunity against the conjugative transfer of a plasmid expressing an RNA protospacer matching spc4 from the L. seeligeri type VI-A CRISPR array (FIG. 5D). Conjugation into nine lysogens was prevented by Cas13a, indicating that the prophages harbored by these lysogens either do not contain or do not express inhibitors of L. seeligeri type VI-A CRISPR-Cas immunity. Only the ΦLS46 lysogen exhibited a high efficiency of plasmid transfer (FIG. 1A), suggesting the possibility that this prophage harbors a Cas13a inhibitor.

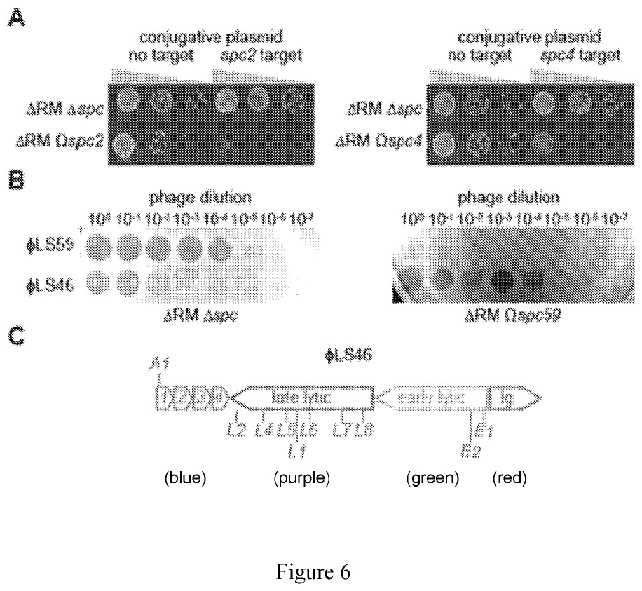

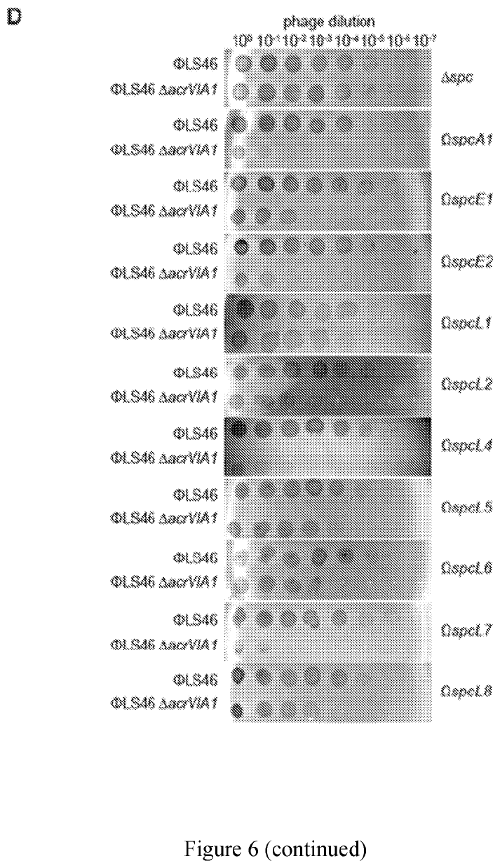

Sequencing of the ΦLS46 genome revealed a similar organization to a previously characterized temperate phage of L. seeligeri, ΦRR4 (14), which harbors four independent transcriptional units: an early lytic region encoding predicted replicases and recombinases involved in phage circularization and genome replication; a late lytic region carrying phage structural genes; a lysogeny cassette containing transcriptional regulators and a predicted site-specific integrase; and a region containing six genes, two of them with homology to the Cas9 inhibitors AcrIIA1 and AcrIIA2, for the evasion of type II CRISPR-Cas immunity by ΦRR4. In ΦLS46, however, this region contains four genes, none of which display strong homology to known inhibitors (FIG. 1B). To investigate if this region contains a type VI Acr, we cloned the operon with its native promoter into a plasmid (pgp1-4), introduced it into wild-type L. seeligeri, and tested for Cas13a-mediated immunity against plasmid conjugation as in FIG. 1A. Indeed, the presence of pgp1-4 allowed plasmid conjugation even in the presence of Cas13a targeting, and cloning of each individual gene allowed us to identify gp2 as the gene responsible for this anti-CRISPR phenotype (FIG. 1C). Accordingly, we renamed gp2 “type VI-A anti-CRISPR 1”, or AcrVIA1. Next we tested if AcrVIA1 was necessary for inhibition of Cas13a during ΦLS46 infection. We created a derivative of the ΔRM Δspc strain in which we ectopically integrated different spacer sequences, with their transcription controlled by the native CRISPR promoter (strain ΔRM ΩspcX, FIG. 5A). First, we inserted spacers targeting transcripts of either a conjugative plasmid or phage ΦLS59, whose genome lacks acr genes, and confirmed that they can provide efficient immunity in this experimental system (FIGS. 6A-B). We then cloned 10 spacers targeting different transcript regions of ΦLS46 (FIG. 6C), none of which conferred immunity (FIG. 6D). Finally, we isolated phage mutants in the Acr region of ΦLS46 and tested the same spacers for immunity against them. While none of the spacers protected against wild-type ΦLS46, both Agp1-4 and ΔacrVIA1 mutants exhibited 1-6 orders of magnitude of sensitivity to Cas13a interference (FIGS. 1D and 6D). We expressed AcrVIA1 using the pgp2 plasmid and found that it inhibited targeting of the Cas13a-susceptible phage ΦLS59 (FIG. 1E). Finally, the inhibition of anti-plasmid immunity observed in the WT(ΦLS46) lysogen was abolished when we performed the conjugation assay using an WT(ΦLS46 ΔacrVIA1) lysogen (FIG. 1A). To explore the effect of AcrVIA1 on the Cas13a-induced host cell dormancy that is fundamental for type VI-A immunity, we induced the expression of spc4 target RNA, which was previously shown to cause a severe growth defect as a result of nonspecific host transcript degradation (14). Expression of the inhibitor using the pgp2 plasmid, however, reverted this growth defect (FIG. 1F).

Collectively, data presented in this disclosure indicate that acrVIA1 is necessary and sufficient to inhibit Cas13a-induced growth arrest and thus thwart type VI CRISPR immunity against plasmids and phages.

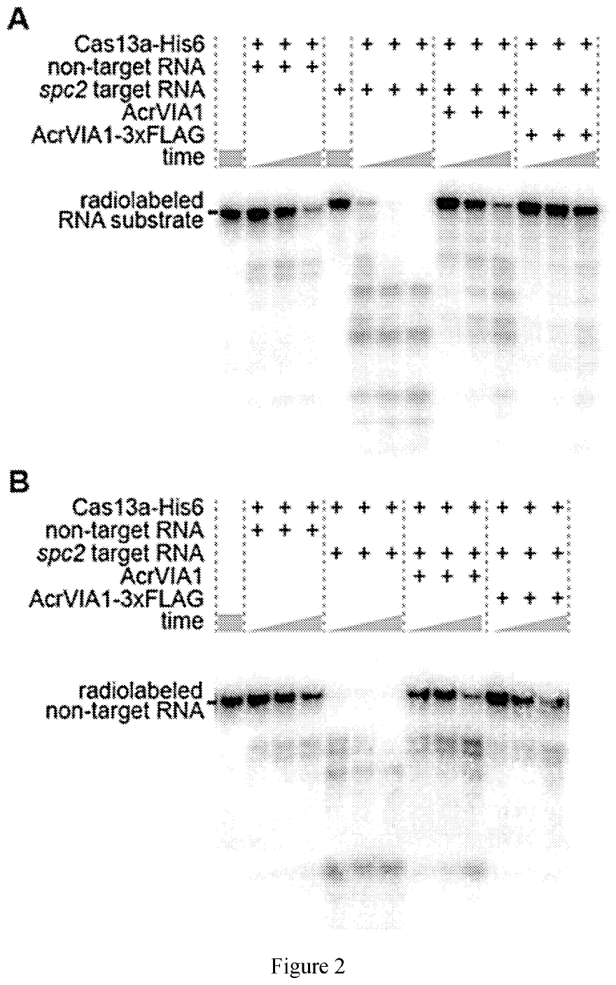

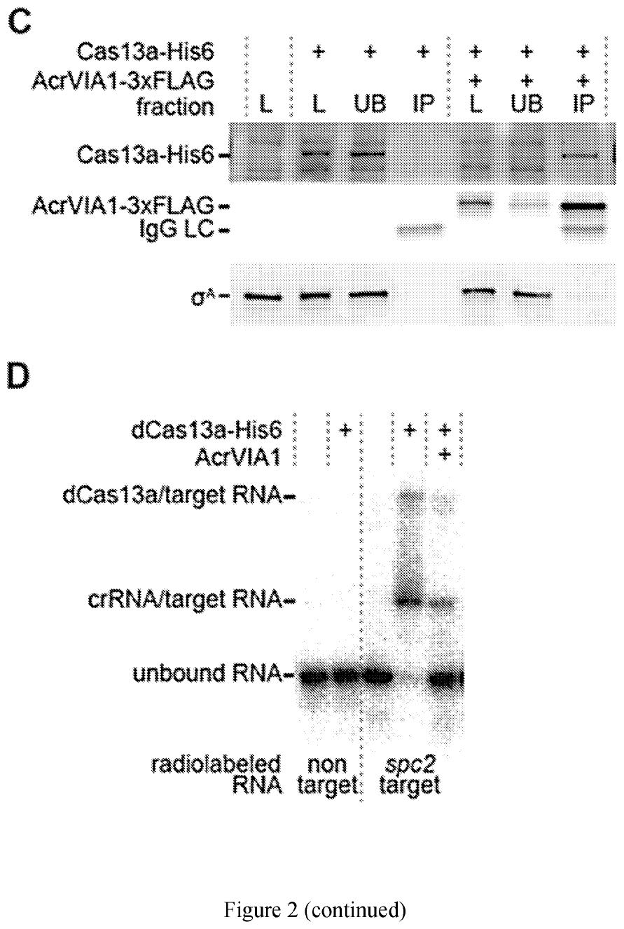

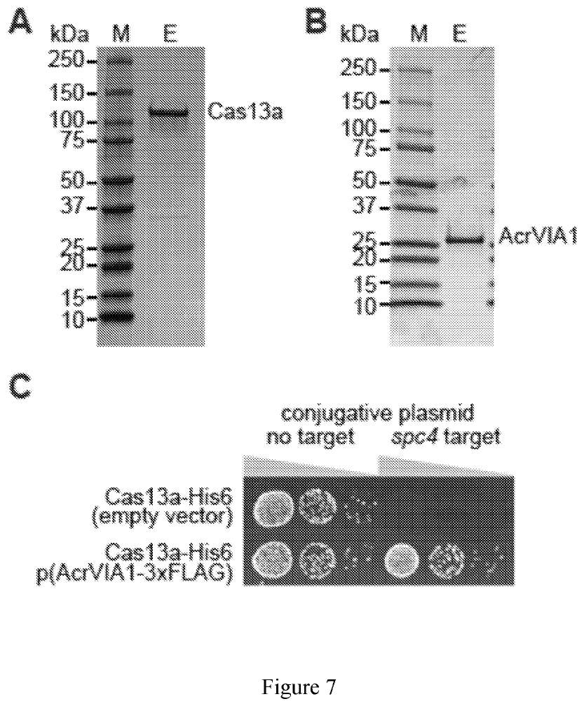

The inhibition of Cas13a-induced growth arrest suggests that AcrVIA1 inhibits the trans-RNase activity of Cas13a. To investigate this, we purified both proteins (FIG. 7A-B) and tested their activities using in vitro RNA protospacer cleavage assays. A radiolabeled target RNA was used to investigate inhibition of Cas13a's cis-RNase activity. Purified L. seeligeri Cas13a^{cRNA} catalyzed rapid RNA cleavage upon addition of protospacer RNA, and this activity was not observed with a non-target RNA (FIG. 2A). In the presence of excess AcrVIA1, however, target RNA cleavage was inhibited. Similarly, AcrVIA1 inhibited Cas13a-mediated trans-cleavage of a labeled non-target RNA upon addition of unlabeled protospacer RNA (FIG. 2B). To investigate the presence of an interaction between the nuclease and its inhibitor, we added C-terminal hexa-histidine and 3xFLAG tags to Cas13a and AcrVIA1, respectively, and confirmed that both are active in vivo, during type VI CRISPR-Cas immunity against plasmid conjugation in L. seeligeri (FIG. 7C) and in vitro (FIG. 2A-B). We expressed Cas13a-His6 either alone or in the presence of AcrVIA1-3xFLAG, then performed immunoprecipitation with anti-Flag antibody resin, and analyzed the input, unbound, and immunoprecipitated fractions by immunoblot using antibodies against His6, FLAG, and the L. seeligeri housekeeping sigma factor σ^{A} (FIG. 2C). In the absence of AcrVIA1-3xFLAG, neither Cas13a-His6 nor σ^{A} co-immunoprecipitated with the anti-FLAG antibody, demonstrating the specificity of the reaction. However, in the presence of AcrVIA1-3xFLAG, we detected both strong immunoprecipitation of tagged AcrVIA1, as well as co-immunoprecipitation of Cas13a-His6. Notably, in the same samples σ^{A} remained unbound to the resin suggesting the presence of a specific interaction of Cas13a with its inhibitor. Finally, we investigated whether the interaction of AcrVIA1 with Cas13a prevents binding of the Cas13a^{cRNA} complex to its complementary target RNA. To test this, we performed an electrophoretic mobility shift assay (EMSA) to measure the association of labeled protospacer RNA with purified Cas13a^{crRNA} complex purified from L. seeligeri (FIG. 2D). To prevent cleavage of the targeted RNA, we introduced mutations in the HEPN domain of Cas13a that eliminate its RNase activity (dCas13a: R445A, H450A, R1016A, H1021A). In the presence of the dCas13a^{crRNA} complex, the majority of the target RNA (but not a non-target RNA) was shifted to multiple higher molecular weight species: one corresponding to a crRNA-protospacer RNA duplex, and higher species representing dCas13a^{crRNA}-protospacer ternary complex. In contrast, in the presence of excess AcrVIA1, the target RNA remained mostly unbound, largely unassociated with dCas13a. Collectively, these results indicate that AcrVIA1 forms a complex with Cas13a^{crRNA} ribonucleoprotein that prevents binding of the complementary target RNA and therefore inhibits both its cis- and trans-RNase activities.

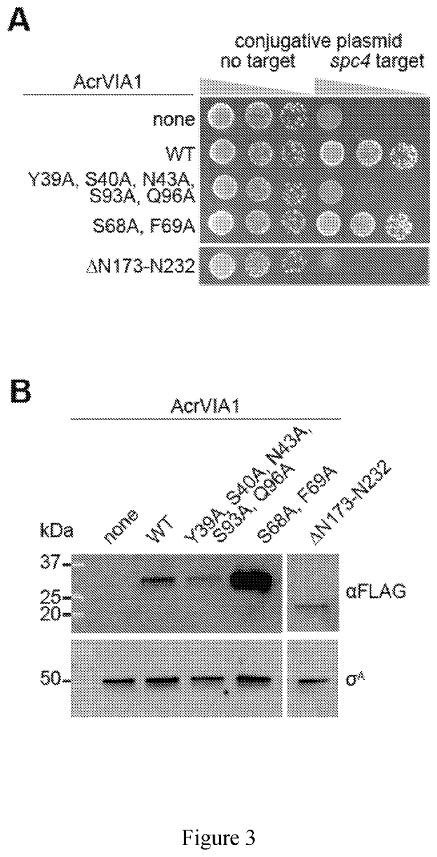

We performed site-directed mutagenesis of the acrVIA1-3xflag (carried by plasmid pgp2) to modify the amino acid residues predicted to mediate the nuclease-inhibitor contacts, and thus test the contribution of different putative interactions to the inhibition of type VI-A immunity against plasmid conjugation. Alanine substitutions at predicted Cas13a^{crRNA}-interacting residues in AcrVIA1 (quintuple mutant Y39A, S40A, N43A, S93A, Q96A) caused nearly complete loss of inhibitory function (FIG. 3A) without affecting protein expression (FIG. 3B). In contrast, the S68A, F69A double mutant retained full function (FIG. 3A), suggesting that the interaction with Cas13a is unperturbed in this mutant. However, the mutation also led to an increase in expression levels (FIG. 3B), which could compensate for a partial loss of function. Finally, deletion of the two C-terminal helices in AcrVIA1 (ΔN173-N232) abrogated inhibition (FIG. 3A) without affecting protein expression (FIG. 3B), indicating that the contacts between these helices and the Cas13a Linker domain play a critical role in Cas13a inhibition.

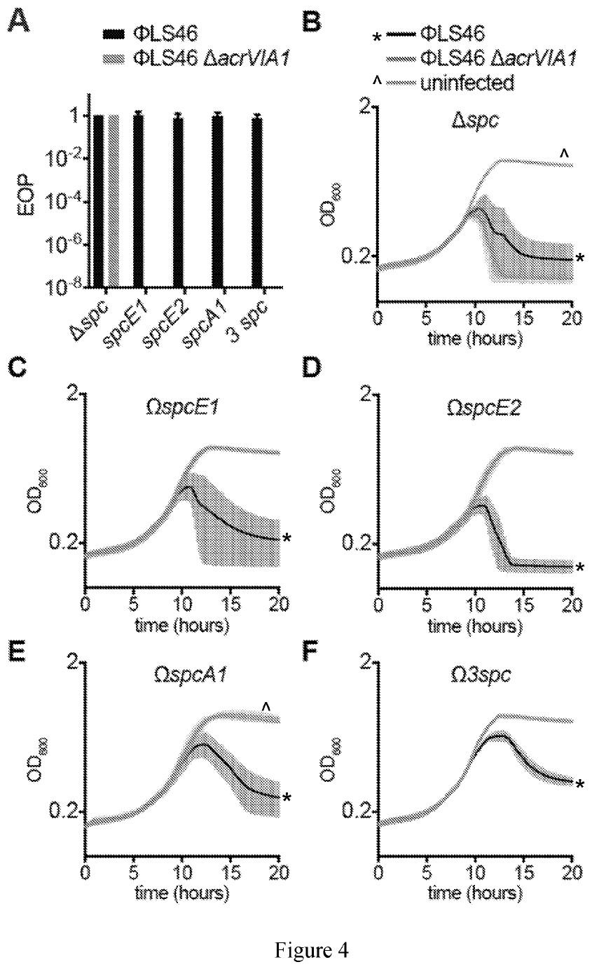

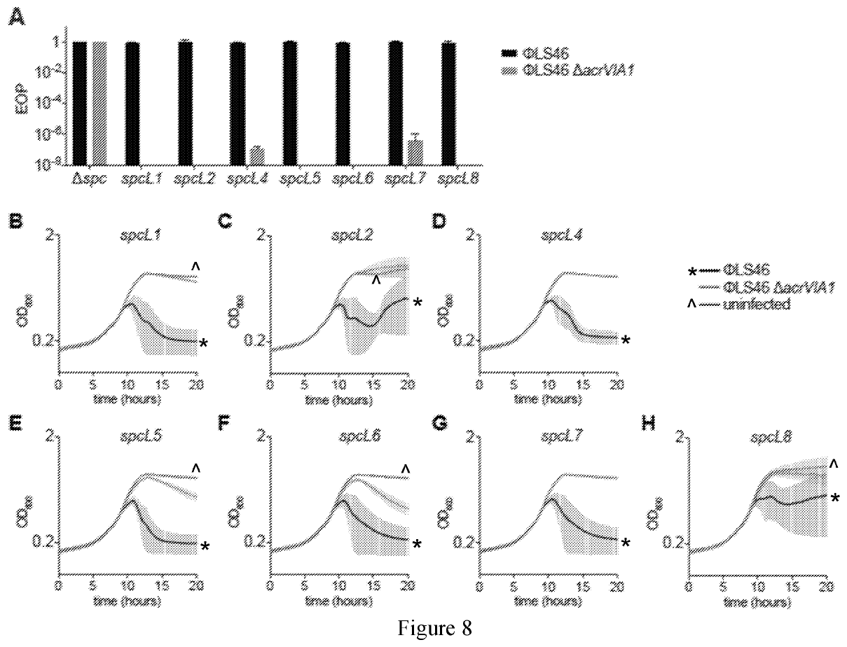

Previously described anti-CRISPRs that inhibit type I and II CRISPR systems require multiple rounds of infection to completely inhibit anti-phage immunity, and fail in conditions of strong CRISPR immunity or low viral load (21, 22). To investigate if AcrVIA1 also displayed limited inhibition capabilities, we first tested its efficacy in conditions of weak or strong type VI CRISPR-Cas immunity, by infecting cells harboring either one or three targeting spacers, respectively (FIG. 1B). As a control we performed infections with the ΦLS46 ΔacrVIA1 mutant phage and measured the efficiency of plaquing (EOP) in the different host backgrounds. When compared to infection of hosts without targeting spacers, all three individual spacers as well as the triple combination provided efficient immunity against this mutant, reducing the EOP by at least eight orders of magnitude, below our limit of detection. In contrast, immunity was completely abrogated (˜100% EOP) during infections with wild-type ΦLS46, when plating on either the single-spacer or triple-spacer strains (FIG. 4A). EOP values are obtained after spotting ten-fold dilutions of the phage stock on both targeting and non-targeting conditions (FIG. 6D) and the lowest dilution contains only a few viral particles. The 100% EOP value obtained indicates that all of these isolated viral particles were able to inhibit Cas13a and form a visible plaque. This result suggests that the dose of AcrVIA1 delivered by a single phage particle inactivates Cas13a with sufficient potency to permit a successful lytic cycle and therefore the inhibitor is effective in conditions of enhanced immunity that normally would prevent inhibition by the type I and II Acrs (21, 22). To test this further, we performed infection of liquid cultures of L. seeligeri ΔRM at an extremely low MOI, 0.000001, using same set of spacers used in the EOP assay. In this experiment, immunity is determined by following the growth measured as OD600 over time. In the absence of a targeting spacer, both wild-type and ΔacrVIA1 mutant phage lead to the lysis of the bacteria in the culture (FIG. 4B). While L. seeligeri strains harboring a single ΦLS46-targeting spacer were immune to the ΔacrVIA1 mutant phage, wild-type ΦLS46 was able to lyse the cultures (FIG. 4C-E), showing that AcrVIA1 efficiently inhibits type VI-A CRISPR immunity even in conditions of low MOI, where previously reported Acrs have been shown to fail. Similar results were obtained with strains harboring other targeting spacers (FIGS. 6C and 8). Infection of the strain containing three targeting spacers resulted in a delay in lysis (FIG. 4F), which is consistent with the stronger immunity provided by the presence of multiple spacers, yet still led to efficient inhibition of the type VI-A CRISPR immune response, further demonstrating that AcrVIA1 can enable viral propagation in conditions that are extremely unfavorable for the success of Cas13a inhibition (low MOI and multiple Cas13a targeting spacers).

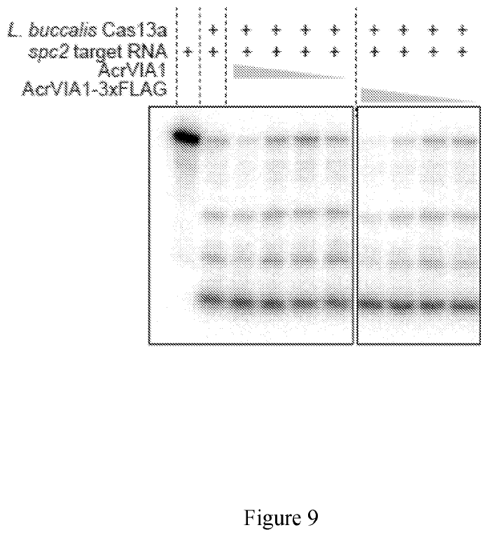

In this disclosure, we used genetic and biochemical approaches to isolate and characterize a phage-encoded inhibitor of L. seeligeri Cas13a: AcrVIA1. This inhibitor interacts with the crRNA-exposed face of Cas13a and makes specific contacts with both the nuclease and its guide RNA that prevent the binding of a complementary target RNA and the conformational changes required for the activation of Cas13a's RNase function. We analyzed whether AcrVIA1 could inhibit other Cas13 family members. Addition of AcrVIA1 had no effect on protospacer RNA cleavage by purified L. buccalis Cas13a^{crRNA} (FIG. 9). Thus, biochemical tests indicate that, as is the case for other Acr types (29, 30), AcrVIA1 is limited to neutralizing only the L. seeligeri type VI-A CRISPR-Cas immune response. Bioinformatic analysis of the other three genes that form the Acr operon of phage ΦLS46 revealed limited similarity between the Gp3 protein and AcrIIA6 from Streptococcus phage DT1, suggesting that it may be an inhibitor of type II-A CRISPR-Cas systems. However, no homology was detected for Gp1 or Gp4, raising the possibility that these proteins could inhibit other CRISPR types. Future research will determine whether there are inhibitors of type VI-B, -C and/or D systems, as well as whether there are other mechanisms of Cas13 inhibition, such as target RNA modification, inhibition of crRNA loading, crRNA guide degradation, post-translation modification of Cas13 or inhibition of Cas13a activation after target binding.

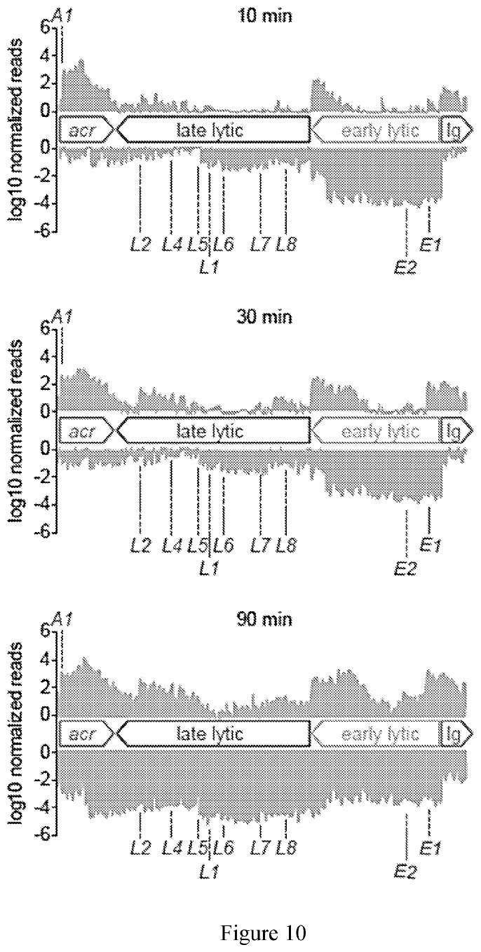

We also found that AcrVIA1 can completely neutralize type VI-A CRISPR-Cas immunity against ΦLS46, even in unfavorable conditions for inhibition such as multiple protospacer targeting and low viral load. We believe this to be a consequence of the lack of phage DNA clearance during the type VI response (14). This would lead to a continuous transcription and translation of AcrVIA1 and progressive neutralization of Cas13a. Assuming that the collateral RNA degradation generated by activation of Cas13a in Listeria hosts allows a low level of AcrVIA1 transcription and translation, enough inhibitor will accumulate to inactivate all the Cas13a molecules inside the bacterial cell. This is in contrast to type I and II Acrs, whose initial production inhibits only a fraction of Cascade-Cas3 and Cas9 molecules, respectively, and the Acr-harboring phage is destroyed by the nucleases that remain active (21, 22). Gradual inhibition of Cas13a after phage infection would require AcrVIA1 to constantly capture the Cas13a^{crRNA} molecules that disengage from the target RNA and prevent them from finding their targets again. Alternatively, the inhibitor could displace the target RNA molecules from activated Cas13a′^{crRNA} nucleases, to de-activate them. Such a mechanism would be especially effective when the target RNA is a transcript that is produced, and therefore activates Cas13a, before AcrVIA1 is generated. Indeed, spcA1-mediated immunity, which targets the first gene of the acr operon (gp1) and should activate Cas13a before production of the inhibitor (encoded by the second gene of the operon, gp2) is effectively abrogated by AcrVIA1. And moreover, many of the spacers used in this disclosure target phage transcripts that are abundantly produced shortly after infection (those targeted by spcA1, spcE1, and spcE2 for example, FIG. 10) and yet are unable to provide Cas13a-mediated immunity in the presence of AcrVIA1. Regardless of the details of the molecular mechanisms of inhibition, the present disclosure demonstrates that AcrVIA1 is likely a useful component of the Cas13 toolbox, allowing control of this nuclease during its RNA editing, RNA knock-down and/or RNA visualization applications (31, 32).

Notwithstanding the foregoing description, the disclosure further comprises use of the described protein inhibitor to improve certain diagnostic assays which are used to analyze RNA, such as in biological samples. In embodiments, the disclosure includes use of the inhibitor in any diagnostic assay that is intended to determine the presence or absence of a particular RNA polynucleotide, and quantitative approaches are also included.

In embodiments, a biological sample analyzed according to this disclosure comprises any suitable biological sample, including but not limited to blood, urine, mucosa, mucosal secretions, saliva, and lacrimal secretions. In embodiments, a biological sample is tested directly. In embodiments, the biological sample is subject to a processing step before testing, a non-limiting example of which comprises RNA extraction. In embodiments, a diagnostic assay of this disclosure may exhibit increased sensitivity to the presence or absence of a particular RNA, and in embodiments may obviate the requirement for cDNA synthesis and amplification and still provide a test with sufficient sensitivity and specificity. Accordingly, in certain embodiments, a diagnostic test of this disclosure may be performed without using reverse transcriptase, and/or may be performed without a PCR amplification step. In embodiments, a diagnostic test of the disclosure may be performed without transcription of a PCR-amplified template.

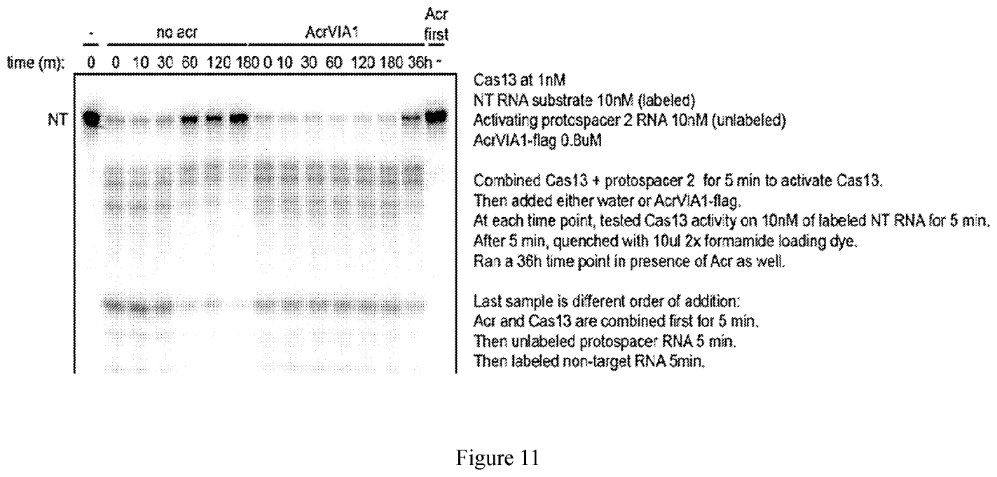

In embodiments, in any diagnostic assay used to detect and/or quantify RNA using a Cas13-related approach, the disclosure includes adding the described inhibitor to biological sample obtained from an individual that is either tested directly, or is processed before testing, such as to separate RNA from the sample. In embodiments, the inhibitor is added a short time (e.g., within 1 second to 60 minutes) after Cas13 in sample has associated with the target RNA, if the target RNA is present, in the patient sample. Without intending to be bound by any particular theory, and as in part illustrated by FIG. 11 and its descriptive text, it is considered that addition of the described inhibitor causes the Cas13 complex with the guide RNA to “lock on” to the Cas13/guide RNA/target RNA complex, which prolongs its non-specific RNA nuclease activity. Accordingly, in certain embodiments, such as for the SHERLOCK assay and adaptations of it as further described below, labeled reporter RNA that is not recognized by the guide RNA may be added before, concurrently, or after the inhibitor is added to the assay. In embodiments, the assay reaction comprises a biological sample, a Cas13, and a guide RNA targeted to an RNA of interest. The presently described inhibitor is then added to extend the longevity of Cas13 cleavage of detectably labeled reporter RNA, but only after the Cas13 is complexed with the targeted RNA, if present, in a guide RNA directed manner. Thus, compositions comprising a Cas13, a guide RNA, a detectably labeled reporter RNA, and a Cas13 inhibitor as described herein are encompassed by the disclosure.

In embodiments, the disclosure includes adding the described inhibitor to an assay that comprises Cas13, and a guide RNA targeted to a particular RNA polynucleotide sequence of interest, and at least one reporter RNA, wherein the reporter RNA is configured to permit Cas13-mediated detection of its degradation, or lack of degradation by the Cas13, e.g., the reporter RNA can be detectably cleaved when the non-specific RNA nuclease activity of Cas13 is triggered.

In non-limiting embodiments, a reporter RNA polynucleotide is not targeted by the Cas13-related guide RNA, and is labeled at one position with a detectable label, and also with a moiety that quenches a detectable signal from the detectable label at another position. In embodiments, a fluorophore and a quencher moiety are conjugated to the reporter RNA in sufficient proximity to one another such that the detectable signal is quenched when the RNA is intact. Accordingly, when and if the RNA reporter is cleaved by the non-specific nuclease activity of the Cas13, which is considered to only become active once the Cas13 has engaged a target in a guide-RNA directed manner, the detectable label is liberated from the intact reporter RNA, and a signal from it can be detected using any suitable approach.

In embodiments, any detectable label can be used with the reporter RNA, non-limiting examples of which include fluorophores, metals or chemiluminescent moieties, fluorescent particles, quantum dots, etc., provided the signal from the detectable label can be quenched, or its intensity shifted to a different wavelength in, for example, a fluorescence resonance energy transfer (FRET) process by a suitable quencher moiety conjugated to the reporter RNA.

In embodiments, an inhibitor of this disclosure is added to an assay such as the so-called SHERLOCK (for Specific High Sensitivity Enzymatic Reporter UnLOCKing) assay, described in PCT publication WO2017219027, published Dec. 21, 2017, and SHERLOCK: nucleic acid detection with CRISPR nucleases, Kellner M J, Koob J G, Gootenberg J S, Abudayyeh O O, and Zhang F. Nature Protocols. 2019, October;14(10):2986-3012. doi: 10.1038/s41596-019-0210-2. (NATURE PROTOCOLS, VOL 14, OCTOBER 2019, 2986-3), the disclosures of each of which are incorporated herein by reference. In embodiments, the SHERLOCK assay is adapted to omit reverse-transcriptase cDNA synthesis and subsequent amplification using PCR-based approaches. In embodiments, less PCR amplification products are required to detect the presence or absence of RNA, relative to a control assay wherein the inhibitor is not included.

In a non-limiting embodiment, the disclosure provides for use of the described inhibitor for detecting RNA viruses, including but not limited to the coronavirus referred to in the art the time of this disclosure as SARS-CoV-2, which causes COVID-19. In an embodiment, the assay is performed using a lateral flow device. In embodiments, the testing is performed by testing for the presence or absence of RNA encoded by the viral S gene and/or the Orflab gene. In embodiments, the Cas13 used in this approach or related approaches is LwaCas13a. In embodiments, liberated label can be detected in the lateral flow device at a predetermined position. Suitable controls may be included, such as a predetermined amount of synthetically produced viral target RNA.

FIG. 11 provides support for use of the Cas13 inhibitor of the present disclosure in diagnostic assays, and to enhance existing assays. In particular, and without intending to be bound by any particular theory, it is considered that in its current form, the SHERLOCK protocol and previous adaptations thereof, require processing and amplification of RNA in the biological sample to be tested. Generally, if the target molecule is RNA and is present in the sample, in order for it to be detected using the protocol available prior to the present disclosure, the RNA must be reverse-transcribed into cDNA. These cDNAs are PCR-amplified, and then transcribed back into RNA, to generate levels of RNA detectable by Cas13.

In the present disclosure, it is revealed that after Cas13 has already engaged target RNA, addition of AcrVIA1 enhances the longevity of Cas13 activity. Thus, it is expected that this aspect of the inhibitor may make Cas13-based diagnostics more sensitive, and may obviate the need for reverse transcription and PCR amplification of patient samples.

In more detail, FIG. 11 provides results showing enhanced Cas13 activity in the presence of the AcrVIA1 inhibitor. Results in FIG. 11 are from an RNA cleavage assay that assesses the nonspecific activity of Cas13 in vitro using purified components. To produce the depicted results, a synthetic nonspecific RNA was radiolabeled is shown in the band labeled NT (non-target). This band is not recognized by Cas13's guide RNA but it is a substrate for Cas13 cleavage. In the first (left most) lane no Cas13 was added, and the labeled RNA substrate is intact. We next performed two reactions: one (labeled “no acr”) contained Cas13 and its guide RNA at 1 nM, plus 10 nM of target RNA (termed protospacer 2) that is recognized by the guide RNA and thus activates Cas13's nonspecific cleavage activity. This reaction proceeded for 5 minutes. At each indicated time point (0, 10, 30, 60, 120, 180 minutes), we analyzed the ability of Cas13 to perform nonspecific cleavage by adding a sample of the reaction to the labeled RNA substrate. In the early time points, Cas13 is very active and cleaves the nonspecific substrate RNA. Over time, Cas13 loses activity, likely because the supply of activating target RNA is exhausted. In the second reaction (“AcrVIA1”), we combined Cas13, guide RNA, and target as in the first, and allowed Cas13 activation to proceed 5 minutes. Then we added an excess of AcrVIA1 protein, and assessed Cas13 activity at the same time points shown for the first reaction. In the presence of AcrVIA1, it can be seen that Cas13 remains “on” throughout the entire time course. Indeed, Cas13 activity is observable 36 hours after initial Cas13 activation. These data support the use of the AcrVIA1 inhibitor to make presently diagnostic tools more sensitive, as discussed above. It should be recognized that the presently demonstrated effect dependent in part on the sequential performance of steps. Specifically, if the AcrVIA1 inhibitor is added to the reaction containing Cas13 before adding the target RNA, then AcrVIA1 prevents target RNA binding and no Cas13 activity is observed (“Acr first” lane) in FIG. 11.

The following materials and methods were used to produce results described in this disclosure.

### METHODS

### Bacterial Strains and Growth Conditions

All genetically modified L. seeligeri strains generated as described herein are derived from L. seeligeri SLCC3954 (23). Environmental L. seeligeri isolates and L. monocytogenes strains are listed in Table S2. Unless otherwise stated, L. seeligeri and L. monocytogenes strains were cultured in Brain Heart Infusion (BHI) medium at 30° C. Where appropriate, BHI was supplemented with the following antibiotics for selection: nalidixic acid (50 μg/mL) chloramphenicol (10 μg/mL), erythromycin (1 μg/mL), or kanamycin (50 μg/mL). For cloning, plasmid preparation, and conjugative plasmid transfer, E. coli strains were cultured in Lysogeny Broth (LB) medium at 37° C. Where appropriate, LB was supplemented with the following antibiotics: ampicillin (100 μg/mL), chloramphenicol (25 μg/mL), kanamycin (50 μg/mL). For conjugative transfer of E. coli—Listeria shuttle vectors, plasmids were purified from Turbo Competent E. coli (New England Biolabs) and transformed into the E. coli conjugative donor strains SM10 λkpir or S17 λpir (33).

### Phage Isolation and Propagation

Temperate listeriophages were isolated by prophage induction via stimulation of the SOS response with the DNA-damaging agent mitomycin C, followed by plaque isolation on the L. seeligeri ΔRM Δspc indicator strain. Each strain of L. seeligeri and L. monocytogenes was cultured overnight and diluted to OD₆₀₀=0.1, then treated with 1 μg/mL mitomycin C to activate the phage lytic cycle. Prophage induction was carried out overnight at 30° C., then culture supernatants were passed through 0.45 μm filters. Each filtrate was screened for phages by infection of ΔRM Δspc using the top agar overlay method: 100 ul of serially diluted induction filtrate was used to infect 100 μL of saturated ΔRM Δspc culture in a 5 mL overlay of BHI containing 0.75% agar, in the presence of 5 mM CaCl₂. Infection plates were incubated at 30° for 24 hrs. Single plaques were resuspended in BHI, then propagated three times on ΔRM Δspc, a single plaque was isolated each time to ensure phage purity. High titer phage lysates were obtained by preparing top agar infections of ΔRM Δspc with plaques at near-confluent density, then soaking the agar with SM buffer (100 mM NaCl, 10 mM MgSO₄, 50 mM Tris-HCl pH 7.5).

### Plasmid Construction and Preparation

All genetic constructs for expression in L. seeligeri were cloned into the following three compatible shuttle vectors, each of which contains an origin of transfer sequence for mobilization by transfer genes of the IncP-type plasmid RP4. These transfer genes are integrated into the genome of the E. coli conjugative donor strains SM10 λpir and S-17 λpir (33). All plasmids used in this disclosure, along with details of their construction, can be found in Table S2. pPL2e—single-copy plasmid conferring erythromycin resistance that integrates into the tRNA^{Arg} locus in the L. seeligeri chromosome (34). pAM8—E. coli—Listeria shuttle vector conferring chloramphenicol resistance (35). pAM326—E. coli—Listeria shuttle vector conferring kanamycin resistance (produced according to this disclosure). To express crRNAs containing engineered spacers, a minimal type VI CRISPR array containing the native promoter and a single repeat-spacer-repeat unit with BsaI entry sites was cloned into BamHI/SalI-digested pPL2e to generate pAM305. To clone new spacers, pAM305 was digested with BsaI, and ligated to spacer inserts consisting of annealed oligos with cohesive overhangs compatible with the sticky ends generated by BsaI-cleavage of pAM305.

All plasmid targeting assays described herein use the pAM8-derived plasmid pAM54 (35), in which a protospacer matching the endogenous type VI spc4 was cloned into the 3′ untranslated region of a chloramphenicol resistance cassette. The negative control for plasmid targeting assays is pAM8, which contains the chloramphenicol cassette without a protospacer.

Putative anti-CRISPR constructs were assembled by cloning into HindIII/EagI-digested pAM326.

### E. coli—L. seeligeri Conjugation

All genetic constructs for expression in L. seeligeri were introduced by conjugation with the E. coli donor strains SM10 λpir, S-17 λpir (33), or for allelic exchange (see below), β2163 ΔdapA (36). Donor cultures were grown overnight in LB medium supplemented with the appropriate antibiotic (25 μg/mL chloramphenicol for pPL2e-derived plasmids, 100 μg/mL ampicillin for pAM8-derived plasmids, or 50 μg/mL kanamycin for pAM326-derived plasmids) at 37° C. Recipient cultures were grown overnight in BHI medium supplemented with the appropriate antibiotic (1 μg/mL erythromycin for pPL2e-derived plasmids, 10 μg/mL chloramphenicol for pAM8-derived plasmids, 50 μg/mL kanamycin for pAM326-derived plasmids) at 30° C. 100 μL each of donor and recipient culture were diluted into 10 mL of BHI medium, and concentrated onto a filter disc (Millipore-Sigma, HAWP04700) using vacuum filtration. Filter discs were laid onto BHI agar supplemented with 8 μg/mL oxacillin (which weakens the cell wall and enhances conjugation) and incubated at 37° C. for 4 hr. Discs were removed, cells were resuspended in 2 mL BHI, and transconjugants were selected on medium containing 50 μg/mL nalidixic acid (which kills donor E. coli but not recipient L. seeligeri) in addition to the appropriate antibiotic for plasmid selection. Transconjugants were isolated after 2-3 days incubation at 30° C.

### Gene Deletions in L. seeligeri

Allelic exchange plasmids were generated by cloning 1 kb homology arms flanking the genomic region to be deleted into the suicide vector pAM215 (14), which does not replicate in Listeria, and contains a chloramphenicol resistance cassette and lacZ from Geobacillus stearothermophilus. These plasmids were then transformed into the E. coli donor strain □2163 AdapA (36), which is auxotrophic for diaminopimelic acid (DAP), selecting on LB medium supplemented with the appropriate antibiotic and 1.2 mM DAP. Conjugation was carried out as described above, except all steps were carried out in the presence of 1.2 mM DAP. Transconjugants were selected on media lacking DAP and containing 50 μg/mL nalidixic acid, to ensure complete killing of donor E. coli, as well as 10 μg/mL chloramphenicol to select for integration of the pAM215-derived plasmid. Chloramphenicol-resistant colonies were patched on BHI supplemented with 100 μg/mL 5-Bromo-4-Chloro-3-Indolyl β-D-Galactopyranoside (X-gal) and confirmed lacZ+ by checking for blue colony color. Plasmid integrants were passaged 3-4 times in BHI at 30° in the absence of antibiotic selection, to permit loss of the integrated plasmid. Cultures were screened for plasmid excision by dilution and plating on BHI+X-gal. White colonies were checked for chloramphenicol sensitivity, then chromosomal DNA was prepared from each, and tested for the desired deletion by PCR using primers flanking the deletion site. Deletions were finally confirmed by Sanger sequencing.

### Bacterial Genome Sequencing, Genome Assembly, and ΦLS46 identification

The ΦLS46 genome was sequenced by whole-genome sequencing and assembly of its parent lysogen, L. seeligeri LS46. Chromosomal DNA was prepared from LS46 by lysozyme digestion of the cell wall, followed by cell lysis with 1% sarkosyl, then phenol-chloroform extraction and ethanol precipitation. 1 ng of chromosomal DNA was used to make an NGS library using the Illumina Nextera XT DNA Library Preparation Kit according to the manufacturer's instructions. Library quality was confirmed by analysis on Agilent

TapeStation, then 2×150 bp paired-end sequencing was carried out on the Illumina NextSeq platform. Raw reads were quality-trimmed using Sickle (github.com/najoshi/sickle) using a quality cutoff of 30 and length cutoff of 45. Trimmed reads were assembled using SPAdes (cab.spbu.ru/software/spades/) with the default parameters, which resulted in 140 assembled contigs with an N50 of 2841899. These contigs were mapped onto the completed reference genome of L. seeligeri SLCC3954 using Medusa (combo.dbe.unifi.it/medusa/) with the default parameters, which resulted in 105 scaffold assemblies. In our draft genome assembly, one scaffold (Scaffold 1) represents a 2.8 Mbp assembly, Scaffold 7 contains 46 Kbp, and each of the remaining 103 scaffolds contains between 100-1300 bp. To identify putative prophages in the assembled genome, we used Phaster (phaster.ca), which predicted a single prophage element, occupying the entirety of Scaffold 7. We confirmed that this prophage (ΦLS46) was the one isolated by mitomycin C induction of LS46 using PCR of the ΔRM Δspc-passaged phage stock with ΦLS46-specific primers.

### Construction of Gene Deletions in ΦLS46

Gene deletions in ΦLS46 were constructed in two ways. One group of deletions was obtained by selection of spontaneous escapers of Cas9 targeting of the anti-CRISPR locus in ΦLS46. A Cas9 spacer targeting the anti-CRISPR region (gp4) was cloned into the vector pAM307, which carries Cas9 from Streptococcus pyogenes along with a repeat-spacer-repeat construct with BsaI entry sites. This plasmid (pAM379) was introduced into ΔRM Δspc, which was then infected with ten-fold serial dilutions of ΦLS46 in a plaque assay on BHI top agar. Cas9-targeting reduced the efficiency of ΦLS46 plaquing by several orders of magnitude, but spontaneous Cas9-resistant escaper plaques were isolated and checked for deletions by PCR using primers flanking the anti-CRISPR locus. The deletions were then precisely mapped by Sanger sequencing. To generate an in-frame deletion of the acrVIA1 gene, we first assembled a homology repair template (pAM386) containing 1 kb homology arms flanking an in-frame deletion of acrVIAL In the deletion construct, the first six and last six codons of acrVIA1 remain, both to avoid Rho-dependent termination of untranslated RNA, as well as to preserve the Shine-Dalgarno sequence for the gp3 gene predicted to be present in the last six codons of acrVIA1. The repair template plasmid was introduced into ΔRM Δspc, this strain was infected with ΦLS46 in BHI top agar (allowing recombinants to be generated), and a phage stock was harvested. A Cas9 spacer targeting acrVIA1 was cloned into pAM307 to generate pAM377 and introduced into ΔRM Δspc. The ΦLS46 stock passaged on ΔRM Δspc carrying the pAM386 repair template was used to infect ΔRM Δspc carrying pAM377, and Cas9-resistant escaper mutants were isolated. Two mutant phage isolates were Sanger sequenced across the acrVIA1 gene, and found to contain the precise deletion.

### In Vitro RNA Cleavage Assays

10 μM synthetic RNA substrates (listed in Table S7) were labeled with ATP [γ-³²P] for 30 min at 37° with 1 ul NEB T4 Polynucleotide Kinase, then purified using GE MicroSpin G-50 columns. In a 10 μL reaction, 1 nM purified L. seeligeri Cas13-His6:crRNA complex was combined with 10 nM synthetic target RNA, in buffer containing 10 mM HEPES pH 7.0, 150 mM NaCl, 5 mM MgCl₂, 5 mM β-mercaptoethanol, and 5% glycerol, at room temperature for the indicated time. Reactions were quenched by addition of an equal volume of loading dye (95% formamide, 14 mM EDTA, 0.025% SDS, 0.04% bromophenol blue, 0.04% xylene cyanol), then denatured by boiling 5 min, then crash cooled on ice for 1 min before loading on denaturing TBE-Urea PAGE gels with 15% acrylamide. Reactions were exposed to phosphoscreen 1 hour and imaged with Beckman Coulter FLA7000IP Typhoon storage phosphorimager.

### Co-Immunoprecipitation

ΔCRISPR strains of L. seeligeri harboring pAM364 (Cas13-his6 cloned into a pPL2e backbone) and pAM395 (Ptet-AcrVIA1-3xFlag cloned into a pAM326 backbone) along with empty vector controls, were cultured in 50 mL BHI supplemented with 50 μg/mL kanamycin and 100 ng/mL aTc at 30° C. until the OD₆₀₀ reached 0.7. 30 mL culture samples were harvested, pelleted by centrifugation at 8,000 rpm for 2min, and frozen at −80° C. Pellets were resuspended in 0.5 mL ice-cold lysis buffer (50 mM HEPES pH 7.0, 200 mM NaCl, 5 mM MgCl₂, 5% glycerol, 1 mg/mL lysozyme, supplemented with Roche cOmplete EDTA-free protease inhibitor cocktail. Samples were incubated at 37° C. for 5 min, then placed on ice and lysed by sonication. Insoluble material was pelleted by centrifugation at 15,000 rpm for 1 hr at 4° C. A “load” sample was harvested, then the remaining soluble fraction was applied to 30 μL of pre-equilibrated ANTI-FLAG M2 Affinity Gel (Millipore-Sigma) for 4 hr at 4° C. The resin was pelleted by centrifugation at 2,000 rpm for 1 min, then the “unbound” sample was harvested. The resin was washed three times by centrifugation and resuspension in 1 mL wash buffer (20 mM HEPES pH 7.0, 200 mM NaCl, 5 mM MgCl₂, 5% glycerol). All wash buffer was then removed, and the resin was resuspended in 40 μL 2× Laemmli SDS-PAGE loading buffer lacking β-mercaptoethanol, and boiled for 5 min. The resin was pelleted and supernatant was harvested as the “IP” sample. 5% β-mercaptoethanol was added to all samples before separation on 4-20% acrylamide SDS-PAGE gels. For immunoblot analysis, proteins were transferred to a methanol-activated PVDF membrane, blocked with 5% nonfat milk, and probed with anti-His6 (Genscript), anti-Flag (Sigma) and anti-σ^{A}_{Bacillus subtilis} (37) primary antibodies, then with horseradish peroxidase-conjugated anti-mouse or anti-rabbit secondary antibodies (Bio-Rad). Proteins were detected using Western Lightning chemiluminescence reagent.

### Electrophoretic Mobility Shift Assay

Synthetic RNA substrates were radiolabeled as described for RNA cleavage assays. In vitro RNP assembly was performed for 30 min in a 10 μL reaction at room temperature in the presence of 5mM HEPES pH 7, 10 mM NaCl, 1 mM BME, 5 mM MgCl₂, 1 μg/mL bovine serum albumin, 10 μg/mL salmon sperm DNA, and 5% glycerol. Labeled RNA substrates were added at a final concentration of 10 nM, dCas13a (R445A, H450A, R1016A, H1021A) at 500 nM, and AcrVIA1 at 1800 nM. Reactions were placed on ice 1 min, then 10 μL of non-denaturing loading dye (25% glycerol, 0.05% xylene cyanol, 0.05% bromophenol blue, 50 mM HEPES pH 7.0) was added, and samples were electrophoretically separated by 10% acrylamide native PAGE at 4° C. Gels were exposed and imaged as described for RNA cleavage assays.

### RNA Sequencing and Analysis

L. seeligeri ΔRM Δspc was infected with ΦLS46 at OD₆₀₀ of 0.5, MOI of 0.1 in BHI medium containing 5 mM CaCl₂ at 30° C. At each time point, 1.5 mL of culture was harvested, pelleted by centrifugation at 8,000 rpm for 2 min, and frozen at −80° C. To harvest RNA, samples were resuspended in 90 μL of RNase-free phosphate-buffered saline containing 2 mg/mL lysozyme, and incubated at 37° C. for 3 min. 10 μL of 10% sarkosyl was immediately added to lyse the cells. 300 μL of TRI Reagent (Zymo Research Direct-Zol RNA Miniprep Plus Kit) was added to each sample, then RNA was prepared according to the manufacturer's instructions, eluting in 50 μL RNase-free water. Ribosomal RNA was removed from 1 μg of purified RNA using the NEBNext rRNA Depletion Kit (Bacteria) according to the manufacturer's instructions. After rRNA removal, samples were concentrated using the Zymo Research RNA Clean and Concentrator-5 Kit according to the manufacturer's instructions, eluting RNA in 6 μL RNase-free water. Libraries were prepared for deep sequencing using the Illumina TruSeq Stranded mRNA Library Preparation Kit, skipping mRNA purification and beginning at the RNA fragmentation step. Quality control of libraries was carried out on an Agilent TapeStation. Paired-end (2×75 bp) sequencing was performed on the NextSeq platform. Raw paired-end reads were mapped to the ΦLS46 genome using Bowtie2 with parameters “very-sensitive” and I=40. Using a custom script, the coverage at each position on the ΦLS46 genome was calculated by tallying a count for each of the positions covered by each mapped read. Read counts at each genomic position were normalized to the total number of reads in each library.

### Protein Expression and Purification

The L. seeligeri type VI CRISPR array alongside Cas13a-His6 or dCas13a-His6 (R445A, H450A, R1016A, H1021A) were cloned into pAM8 as described in Table S4, and conjugated into L. seeligeri Δspc Δcas13a. For expression, these strains were cultured at 30° C. in BHI supplemented with 10 μg/mL chloramphenicol for ˜24 hr. Cells were harvested by centrifugation and resuspended in lysis buffer (20 mM Tris-HCl, pH 7.5, 300 mM NaCl, 5% glycerol, 20 mM imidazole, 7 mM β-mercaptoethanol). The harvested cells were then lysed by an EmulsiFlex-C3 homogenizer (Avestin) and centrifuged at 20,000 rpm for 30 min in a JA-20 fixed angle rotor (Avanti J-E series centrifuge, Beckman Coulter). The supernatant was applied to 5 mL HisPur™ Cobalt Resin (Thermo Fisher Scientific). The protein was eluted with lysis buffer supplemented with 500 mM imidazole after washing the column with 10 column volumes of lysis buffer. The elution fractions were further dialyzed against buffer A (20 mM Tris-HCl, pH 7.5, 100 mM NaCl, 7 mM β-mercaptoethanol), and applied on a 1 mL HiTrap SP Fast flow column (GE Healthcare). Proteins were eluted by a linear gradient from 100 mM to 1 M NaCl in 20 column volumes, and then concentrated in 50 kDa molecular mass cut-off concentrators (Amicon) before further purification over a Superdex 200 increase 10/300 GL column (GE Healthcare) pre-equilibrated in buffer B (20 mM Tris, pH 7.5, 150 mM NaCl, 2 mM DTT).

AcrVIA1 was cloned into a pRSF-Duet-1 vector (Novagen), in which the acrVIA1 gene was attached with N-terminal His6-SUMO tag following an ubiquitin-like protease (ULP1). The vector was transformed into Escherichia coli BL21 (DE3) strain and expressed by induction with 0.25 mM isopropyl-β-D-1-thiogalactopyranoside (GoldBio) at 16° C. for 20 hr. Cells were harvested by centrifugation and resuspended in lysis buffer (20 mM Tris-HCl, pH 7.5, 500 mM NaCl, 5% glycerol, 20 mM imidazole, 7 mM β-mercaptoethanol). The harvested cells were then lysed by an EmulsiFlex-C3 homogenizer (Avestin) and centrifuged at 20,000 rpm for 30 min in a JA-20 fixed angle rotor (Avanti J-E series centrifuge, Beckman Coulter). The supernatant was applied to 5 mL HisTrap Fast flow column (GE Healthcare). The protein was eluted with lysis buffer supplemented with 500 mM imidazole after washing the column with 10 column volumes of lysis buffer and 2 column volumes of lysis buffer supplemented with 40 mM imidazole. The elution fractions were further dialyzed against buffer A (20 mM Tris-HCl, pH 7.5, 100 mM NaCl, 7 mM β-mercaptoethanol), and applied on a 5 mL HiTrap Q Fast flow column (GE Healthcare). Proteins were eluted by a linear gradient from 100 mM to 1 M NaCl in 20 column volumes, and then concentrated in 10 kDa molecular mass cut-off concentrators (Amicon) before further purification over a Superdex 200 increase 10/300 GL column (GE Healthcare) pre-equilibrated in buffer B (20 mM Tris, pH 7.5, 150 mM NaCl, 2 mM DTT). Leptotrichia buccalis Cas13 purification was conducted as previously described (35), and the same samples were used in this disclosure.

TABLE S2
Bacterial strains used in this disclosure.

| Species | Strain | Alias | Genotype |
|---|---|---|---|
| L. seeligeri | LS1 | wild-type | wild type |
| L. seeligeri | LS2 | RR4 | wild type |
| L. seeligeri | LS3 | FSL N1-067 | wild type |
| L. seeligeri | LS4 | FSL H6-011 | wild type |
| L. seeligeri | LS5 | FSL H6-169 | wild type |
| L. seeligeri | LS6 | ATCC 51334 | wild type |
| L. seeligeri | LS7 | ATCC 51335 | wild type |
| L. seeligeri | LS8 | FSL S4-0171 | wild type |
| L. seeligeri | LS9 | FSL B8-0099 | wild type |
| L. seeligeri | LS10 | FSL C7-0024 | wild type |
| L. seeligeri | LS11 | FSL S10-0305 | wild type |
| L. seeligeri | LS12 | FSL B8-0287 | wild type |
| L. seeligeri | LS13 | FSL C7-0049 | wild type |
| L. seeligeri | LS14 | FSL H6-0007 | wild type |
| L. seeligeri | LS15 | FSL C7-0030 | wild type |
| L. seeligeri | LS16 | FSL S4-0116 | wild type |
| L. seeligeri | LS17 | FSL S10-0823 | wild type |
| L. seeligeri | LS18 | FSL B8-0050 | wild type |
| L. seeligeri | LS19 | FSL C7-0481 | wild type |
| L. seeligeri | LS20 | FSL T4-0118 | wild type |
| L. seeligeri | LS21 | FSL S4-0037 | wild type |
| L. seeligeri | LS22 | FSL C7-0115 | wild type |
| L. seeligeri | LS23 | FSL S4-0039 | wild type |
| L. seeligeri | LS24 | FSL S10-1784 | wild type |
| L. seeligeri | LS25 | FSL B8-0253 | wild type |
| L. seeligeri | LS26 | FSL C7-0156 | wild type |
| L. seeligeri | LS27 | FSL S10-0300 | wild type |
| L. seeligeri | LS28 | FSL C7-0082 | wild type |
| L. seeligeri | LS29 | FSL L5-0045 | wild type |
| L. seeligeri | LS30 | FSL S10-0030 | wild type |
| L. seeligeri | LS31 | FSL F6-1136 | wild type |
| L. seeligeri | LS32 | FSL L5-0086 | wild type |
| L. seeligeri | LS33 | FSL S10-1611 | wild type |
| L. seeligeri | LS34 | FSL R9-8498 | wild type |
| L. seeligeri | LS35 | FSL C7-0251 | wild type |
| L. seeligeri | LS36 | FSL M6-0039 | wild type |
| L. seeligeri | LS37 | FSL S10-2970 | wild type |
| L. seeligeri | LS38 | FSL C7-0134 | wild type |
| L. seeligeri | LS39 | FSL C7-0462 | wild type |
| L. seeligeri | LS40 | FSL S10-1769 | wild type |
| L. seeligeri | LS41 | FSL H6-0027 | wild type |
| L. seeligeri | LS42 | FSL R8-6874 | wild type |
| L. seeligeri | LS43 | FSL S4-0544 | wild type |
| L. seeligeri | LS44 | FSL S4-0616 | wild type |
| L. seeligeri | LS45 | FSL S4-0939 | wild type |
| L. seeligeri | LS46 | FSL C7-0218 | wild type |
| L. seeligeri | LS47 | FSL C7-0499 | wild type |
| L. seeligeri | LS48 | FSL S6-0001 | wild type |
| L. seeligeri | LS49 | FSL L5-0058 | wild type |
| L. seeligeri | LS50 | FSL A5-0405 | wild type |
| L. seeligeri | LS51 | FSL C7-0081 | wild type |
| L. seeligeri | LS52 | FSL L5-0018 | wild type |
| L. seeligeri | LS53 | FSL R2-0626 | wild type |
| L. seeligeri | LS54 | FSL R8-7055 | wild type |
| L. seeligeri | LS55 | FSL C7-0498 | wild type |
| L. seeligeri | LS56 | FSL R9-4405 | wild type |
| L. seeligeri | LS57 | FSL S10-0788 | wild type |
| L. seeligeri | LS58 | FSL C7-0167 | wild type |
| L. seeligeri | LS59 | FSL M6-0259 | wild type |
| L. seeligeri | LS60 | FSL S10-0889 | wild type |
| L. seeligeri | LS61 | FSL B8-0378 | wild type |
| L. seeligeri | LS62 | N/A | wild type |
| L. seeligeri | LS1 | N/A | Δspc Δcas13a |
| L. seeligeri | LS1 | N/A | ΔRM, Δspc |
| L. seeligeri | LS1 | | 94::Ptet-spc4 |
| L. seeligeri | LS1 | | Δspc Δcas13a, |
| | | | 94°::Ptet-spc4 |
| E. coli | BL21(DE3)- | N/A | F- ompT hsdSB(rB- mB-) |
| | Rosetta2 | | gal dcm (DE3) pRARE2 |
| | | | (CamR) |
| E. coli | SM10 | | thi thr leu tonA lacY |
| | | | supE recA::RP4-2-Tc::Mu |
| | | | Km λpir |
| E. coli | β2163 | | dapA::erm-pir116 |
| | | | RP4-2-Tc::Mu Km |

TABLE S3
Phages used in this Disclosure

| Phage | Host | Genotype | Origin |
|---|---|---|---|
| ϕLS3 | L. seeligeri | wild type | This Disclosure, induced from L. seeligeri LS3 |
| ϕLS4 | L. seeligeri | wild type | This Disclosure, induced from L. seeligeri LS4 |
| ϕLS6 | L. seeligeri | wild type | This Disclosure, induced from L. seeligeri LS6 |
| ϕLS10 | L. seeligeri | wild type | This Disclosure, induced from L. seeligeri LS10 |
| ϕLS14 | L. seeligeri | wild type | This Disclosure, induced from L. seeligeri LS14 |
| ϕLS46 | L. seeligeri | wild type | This Disclosure, induced from L. seeligeri LS46 |
| ϕLS48 | L. seeligeri | wild type | This Disclosure, induced from L. seeligeri LS48 |
| ϕLS51 | L. seeligeri | wild type | This Disclosure, induced from L. seeligeri LS51 |
| ϕLS57 | L. seeligeri | wild type | This Disclosure, induced from L. seeligeri LS57 |
| ϕLS59 | L. seeligeri | wild type | This Disclosure, induced from L. seeligeri LS59 |
| ϕLS62 | L. seeligeri | wild type | This Disclosure, induced from L. seeligeri LS62 |
| ϕEGDe | L. seeligeri | wild type | This Disclosure, induced from L. monocytogenes |
| | | | EGDe |
| ϕ10403S | L. seeligeri | wild type | This Disclosure, induced from L. monocytogenes |
| | | | 10403S |
| U153 | L. seeligeri | wild type | Loessner et al., 2000 Mol. Microbiol |
| A118 | L. seeligeri | wild type | Loessner et al., 2000 Mol. Microbiol |
| ϕLS46 | L. seeligeri | Δgp1-4 | This Disclosure, escaper of pAM379 targeting |
| ϕLS46 | L. seeligeri | ΔacrVIA1 | This Disclosure, pAM386-recombinant escaper of |
| | | | pAM377 targeting |

TABLE S4
Plasmids used in this disclosure.

| Plasmid | Description | Source | Construction Notes |
|---|---|---|---|
| pAM8 | E. coli-Listeria shuttle vector | Meeske, et al. | N/A |
| | (cat) | 2018 Mol Cell | |
| pAM52 | spc2 cat target in pAM8 | Meeske, et al. | N/A |
| | | 2018 Mol Cell | |
| pAM54 | spc4 cat target in pAM8 | Meeske, et al. | N/A |
| | | 2018 Mol Cell | |
| pAM326 | E. coli-Listeria shuttle vector | This Disclosure | Gibson assembly of kanR |
| | (kan) | | (oAM1055/1056), pSK1 ori |
| | | | (oAM1057/1058), oriT |
| | | | (oAM1059/1060) |
| pPL2e | Listeria integrating vector | Lauer, et al. 2002 | N/A |
| | (erm) | J Bacteriol. | |
| pAM215 | lacZ suicide vector (cat) | Meeske, et al. | N/A |
| | | 2019 Nature | |
| pAM305 | Type VI RSR in pPL2e with | This Disclosure | Gibson assembly of BamHI/SalI |
| | BsaI sites for spacer cloning | | digested pPL2e and synthetic |
| | | | fragment 1 (oAM462/451) |
| pAM375 | pgp1-4 | This Disclosure | Gibson assembly of HindIII/EagI |
| | | | digested pAM326 and gp1-4 region |
| | | | (oAM1176/1177) |
| pAM388 | pgp1 | This Disclosure | Gibson assembly of HindIII/EagI |
| | | | digested pAM326, Ptet |
| | | | (oAM1107/572) and gp1 |
| | | | (oAM1178/1179) |
| pAM383 | pgp2 | This Disclosure | Gibson assembly of HindIII/EagI |
| | | | digested pAM326, Ptet |
| | | | (oAM1107/572) and gp2 |
| | | | (oAM1180/1181) |
| pAM384 | pgp3 | This Disclosure | Gibson assembly of HindIII/EagI |
| | | | digested pAM326, Ptet |
| | | | (oAM1107/572) and gp3 |
| | | | (oAM1182/1183) |
| pAM385 | pgp4 | This Disclosure | Gibson assembly of HindIII/EagI |
| | | | digested pAM326, Ptet |
| | | | (oAM1107/572) and gp4 |
| | | | (oAM1184/1185) |
| pAM449 | spc2 in pAM305 | This Disclosure | Ligation of BsaI-digested pAM305 |
| | | | and annealed oAM464/465 |
| pAM450 | spc4 in pAM305 | This Disclosure | Ligation of BsaI-digested pAM305 |
| | | | and annealed oAM770/771 |
| pAM422 | spcA1 targeting ϕLS46 in | This Disclosure | Ligation of BsaI-digested pAM305 |
| | pAM305 | | and annealed oAM1270/1271 |
| pAM380 | spcE1 targeting ϕLS46 in | This Disclosure | Ligation of BsaI-digested pAM305 |
| | pAM305 | | and annealed oAM1192/1193 |
| pAM381 | spcE2 targeting ϕLS46 in | This Disclosure | Ligation of BsaI-digested pAM305 |
| | pAM305 | | and annealed oAM1194/1195 |
| pAM382 | spcL1 targeting ϕLS46 in | This Disclosure | Ligation of BsaI-digested pAM305 |
| | pAM305 | | and annealed oAM1196/1197 |
| pAM434 | spcL2 targeting ϕLS46 in | This Disclosure | Ligation of BsaI-digested pAM305 |
| | pAM305 | | and annealed oAM1370/1371 |
| pAM436 | spcL4 targeting ϕLS46 in | This Disclosure | Ligation of BsaI-digested pAM305 |
| | pAM305 | | and annealed oAM1374/1375 |
| pAM437 | spcL5 targeting ϕLS46 in | This Disclosure | Ligation of BsaI-digested pAM305 |
| | pAM305 | | and annealed oAM1376/1377 |
| pAM438 | spcL6 targeting ϕLS46 in | This Disclosure | Ligation of BsaI-digested pAM305 |
| | pAM305 | | and annealed oAM1378/1379 |
| pAM439 | spcL7 targeting ϕLS46 in | This Disclosure | Ligation of BsaI-digested pAM305 |
| | pAM305 | | and annealed oAM1382/1383 |
| pAM440 | spcL8 targeting ϕLS46 in | This Disclosure | Ligation of BsaI-digested pAM305 |
| | pAM305 | | and annealed oAM1384/1385 |
| pAM442 | Array of 3 spc (spcA1, | This Disclosure | Gibson assembly of BamHI/SalI |
| | spcE1, spcE2) targeting | | digested pPL2e and synthetic |
| | ϕLS46 in pAM305 | | fragment 2 |
| pAM365 | spc59 targeting ϕLS59 in | This Disclosure | Ligation of BsaI-digested pAM305 |
| | pAM305 | | and annealed oAM559/560 |
| pAM386 | repair template for acrVIA1 | This Disclosure | Gibson assembly of HindIII/EagI |
| | deletion | | digested pAM326 and Δgp2 |
| | | | upstream (oAM1176/1206) and |
| | | | Δgp2 downstream (oAM1207/1177) |
| pAM307 | type II CRISPR system (S. | Meeske, et al. | N/A |
| | pyogenes) in pAM8 with | 2019 Nature | |
| | SapI sites for spacer cloning | | |
| pAM377 | Cas9 spc targeting gp4 of | This Disclosure | Ligation of SapI-digested pAM307 |
| | ϕLS46 in pAM307 | | and annealed oAM1186/1187 |
| pAM379 | Cas9 spc targeting gp2 of | This Disclosure | Ligation of SapI-digested pAM307 |
| | ϕLS46 in pAM307 | | and annealed oAM1190/1191 |
| pAM364 | CRISPR array + Cas13-His6 | This Disclosure | Gibson assembly of BamHI/SalI |
| | in pPL2e | | digested pPL2e and CRISPR locus |
| | | | (oAM462/111) |
| pAM395 | AcrVIA1-3xFlag in | This Disclosure | Gibson assembly of HindIII/EagI |
| | pAM326 | | digested pAM326 and synthetic |
| | | | fragment 3 |
| pAM417 | CRISPR array + Cas13-His6 | This Disclosure | Gibson assembly of BamHI/EagI |
| | in pAM8 (for purification) | | digested pAM8 and CRISPR locus |
| | | | (oAM631/1260) |
| pAM419 | CRISPR array + dCas13- | This Disclosure | Gibson assembly of BamHI/EagI |
| | His6 in pAM8 (for | | digested pAM8 and CRISPR locus |
| | purification) | | (oAM631/1260, amplified from |
| | | | dCas13 strain) |
| pAM452 | AcrVIA1(Y39A, S40A, | This Disclosure | Gibson assembly of HindIII/EagI |
| | N43A, S93A, Q96A)-3xFlag | | digested pAM326 and synthetic |
| | in pAM326 | | fragment 4 |
| pAM453 | AcrVIA1(ΔE131-E134)- | This Disclosure | Gibson assembly of HindIII/EagI |
| | 3xFlag in pAM326 | | digested pAM326 and synthetic |
| | | | fragment 5 |
| pAM454 | AcrVIA1(I2A, Y4A)-3xFlag | This Disclosure | Gibson assembly of HindIII/EagI |
| | in pAM326 | | digested pAM326 and synthetic |
| | | | fragment 6 |
| pAM455 | AcrVIA1(S68A, F69A)- | This Disclosure | Gibson assembly of HindIII/EagI |
| | 3xFlag in pAM326 | | digested pAM326 and synthetic |
| | | | fragment 7 |
| pAM456 | AcrVIA1(ΔN173-N232)- | This Disclosure | Gibson assembly of HindIII/EagI |
| | 3xFlag in pAM326 | | digested pAM326 and synthetic |
| | | | fragment 8 |

TABLE S5
Oligonucleotide primers used in this disclosure. The sequences
in each box are single, contiguous sequences.

| Primer | Sequence | SEQ ID NO |
|---|---|---|
| oAM224 | GGCGTGAAAATCAACGACCC | 3 |
| oAM225 | TTTGCTTCAATGTCGCCAGC | 4 |
| oAM451 | GTACCGGGCCCCCCCTCGAGGTTTTGTGATGCATGA | 5 |
| | TTTGTTCTG | |
| OAM462 | CGGCCGCTCTAGAACTAGTGAGTGCCAAGTAACTG | 6 |
| | TGC | |
| oAM464 | AAACTTAGTCAACCCCTCGCTGCATTTTCACATT | 7 |
| oAM465 | TTACAATGTGAAAATGCAGCGAGGGGTTGACTAA | 8 |
| oAM559 | TTACAAAAAGAAGCTAAAGAAGTAAAAGAAGAAG | 9 |
| oAM560 | AAACCTTCTTCTTTTACTTCTTTAGCTTCTTTTT | 10 |
| oAM572 | GTTTAACTCACTCTATCAATGATAGAGAGCTTATTT | 11 |
| | TAATTAT | |
| oAM631 | GCAAGACGTAGCCCAGCGCGTCAGTGCCAAGTAAC | 12 |
| | TGTGC | |
| oAM770 | AAACCATATTTCCAAACTCCACTTTGACTACACC | 13 |
| oAM771 | TTACGGTGTAGTCAAAGTGGAGTTTGGAAATATG | 14 |
| oAM1055 | CCCAGCGAACCATTTGAGG | 15 |
| oAM1056 | TTATGCATCCCTTAACTTAAAACAATTCATCCAGTA | 16 |
| | AAATATAATATTTTATTTTCTCC | |
| oAM1057 | TACTGGATGAATTGTTTTAAGTTAAGGGATGCATAA | 17 |
| | ACTGCATC | |
| oAM1058 | ATCCATGGCCTGGATCCATCAAGCTTAAAATTAGTA | 18 |
| | TAATTATAGCACGAGCTCTGAT | |
| oAM1059 | GCTTGATGGATCCAGGCCATGGATGGCGGCCGCCT | 19 |
| | CTCGCCTGTCCCCTC | |
| oAM1060 | ACCTCAAATGGTTCGCTGGGTTAATCGCTTGCCCTC | 20 |
| | ATCTGT | |
| oAM1107 | CTATAATTATACTAATTTTACATCACGGAAAAAGGT | 21 |
| | TATGC | |
| oAM1111 | TACCGGGCCCCCCCTCGAGGTTAATGATGATGGTGG | 22 |
| | TGATGCTTCATCGTTAATAGCGTTCTTACTAG | |
| oAM1176 | CGTGCTATAATTATACTAATTTTATATATTCGTTGAC | 23 |
| | TACATTTTTCTACTATAATAGAAG | |
| oAM1177 | TGAGGGGACAGGCGAGAGGCTATTACATTACAGCT | 23 |
| | AGTGATAAGTATGTACAG | |
| oAM1178 | CATTGATAGAGTGAGTTAAACACTTTACAAGTTTAA | 24 |
| | CATATTATGTTAATATATAAATATAGC | |
| oAM1179 | TGAGGGGACAGGCGAGAGGCGCCCATTTATTATTT | 25 |
| | GTTATATTTGTTGTAAAAATTTAC | |
| oAM1180 | ATTGATAGAGTGAGTTAAACCTATAGGAGGAAAAA | 26 |
| | ACGATGATCTAC | |
| oAM1181 | GAGGGGACAGGCGAGAGGCTTAATTTAGCTCCTCT | 27 |
| | TTTAAAATTTGTTTGC | |
| oAM1182 | CATTGATAGAGTGAGTTAAACTAAAAGAGGAGCTA | 28 |
| | AATTAAATGACAAATTTAATC | |
| oAM1183 | GAGGGGACAGGCGAGAGGCATTTATATAAAAAGTT | 29 |
| | TAAATTTCTGCATTAAATTCTTGG | |
| oAM1184 | CATTGATAGAGTGAGTTAAACTTTAGGAGGAATTA | 30 |
| | AAATGAATAAATTTGCAT | |
| oAM1185 | TGAGGGGACAGGCGAGAGGCCTGATGTATTATATT | 31 |
| | AATCCTTGCTCTTTTTTATC | |
| oAM1186 | AAACCAAGGTAAATTTGAAGTACAGATTCAAAAA | 32 |
| oAM1187 | AACTTTTTGAATCTGTACTTCAAATTTACCTTGG | 33 |
| oAM1190 | AAACTCATTTCTTTTGATTTCTAAAAATATAGTA | 34 |
| oAM1191 | AACTACTATATTTTTAGAAATCAAAAGAAATGAG | 35 |
| oAM1192 | TTACAACAAATATGGAAAGTAATTTATTTAAATT | 36 |
| oAM1193 | AAACAATTTAAATAAATTACTTTCCATATTTGTT | 37 |
| oAM1194 | TTACTTTATTCGATAAAGACAGCACGAATAAAAA | 38 |
| oAM1195 | AAACTTTTTATTCGTGCTGTCTTTATCGAATAAA | 39 |
| oAM1196 | TTACCAACTATCGAAATTGATTGGAAAATAAATA | 40 |
| oAM1197 | AAACTATTTATTTTCCAATCAATTTCGATAGTTG | 41 |
| oAM1206 | CTCCTCTTTTAAAATTTGTTTGTAGATCATCGTTTTT | 42 |
| | TCCTCCTATAGTC | |
| oAM1207 | CGATGATCTACAAACAAATTTTAAAAGAGGAGCTA | 43 |
| | AATTAAATG | |
| oAM1260 | GCGACCACACCCGTCCTGTGTTAATGATGATGGTGG | 44 |
| | TGATGCTTCATCGTTAATAGCGTTCTTACTAG | |
| oAM1270 | TTACGATGATTAAAATGATGACTGAAAAACAAAA | 45 |
| oAM1271 | AAACTTTTGTTTTTCAGTCATCATTTTAATCATC | 46 |
| oAM1370 | TTACTACAAACAACACGTATAAAAACAAAAAATT | 47 |
| oAM1371 | AAACAATTTTTTGTTTTTATACGTGTTGTTTGTA | 48 |
| oAM1374 | TTACTAATAGAAGAGGTTGTAAAAGATTGTAAAG | 49 |
| oAM1375 | AAACCTTTACAATCTTTTACAACCTCTTCTATTA | 50 |
| oAM1376 | TTACTTTATTACAATATATTCCGCAAACATTCAA | 51 |
| oAM1377 | AAACTTGAATGTTTGCGGAATATATTGTAATAAA | 52 |
| oAM1378 | TTACTTGCAAAAAGAAATTACAAAGGACTTTCATT | 53 |
| oAM1379 | AAACAATGAAAGTCCTTTGTAATTTCTTTTTGCAA | 54 |
| oAM1382 | TTACGAATCTGGACATTTAATTGATTTTGCAAAA | 55 |
| oAM1383 | AAACTTTTGCAAAATCAATTAAATGTCCAGATTC | 56 |
| oAM1384 | TTACTAAAGCAATAGCGAAATACATTGAAGAAAA | 57 |
| oAM1385 | AAACTTTTCTTCAATGTATTTCGCTATTGCTTTA | 58 |
| oAM1415 | GTTTGCCTAAAAATGCGCTTAAATCAGC | 59 |
| oAM1416 | CGTCGTGCAATGCTAATCAAGATTGC | 60 |

TABLE S6
Synthetic gene fragments used in this disclosure

| # | Sequence |
|---|---|
| 1 | ccgggccccccctcgaggTTTTGTGATGCATGATTTGTTCTGTATTATCTTGCATTTCA |
| | TTTTCATAAACTAACTTGCCCCCGTTTTTATCCCTAGAAATTAGTACTTTTTTT |
| | CTATCAACCTCTACTTTAGTAATTCTCATAGTTTTCACCTCAATGATTTTTTTC |
| | TCTCTTCTATTGTACATATAATCACAAAAAAATAAAACACCTAAATGATGGA |
| | TAAGCGTTTTTATACTTATCCAcatTAGACGTTTTAGTCCTCTTTCATATAGAG |
| | GTAGTCTCTTACTGAGACCAGTCTCGGAAGCTCAAAGGTCTCAGTTTTAGTC |
| | CTCTTTCATATAGAGGTAGTCTCTTACCCTACTTAATAATAGTAATTAAAACA |
| | ACCAATGTAAAGGATATAATCAATATATTTAAAGTTTGCACGAGAATGCAAT |
| | CATTTTATTCATAAATATCATATCATTTATAAGCTCTATTTTCCATTTTCTAAG |
| | GCTAATAAATAAAACTGCTGTACCTATGGATCTAAGGAAGACTTATGCACAC |
| | AGTACAGCAACTTTTCAGCATGATTTGTGTTAAAAACATTTAATTTATTGTAG |
| | CAATTTCTTGAGAATCTATAAACCATTTTCCGGTTTCAATTTTAACCAACTCC |
| | AAATCTAACAATACTTGATTTTCGGTTTTCTTTTGATTGTCGACTTGAAAAGC |
| | AGACCAGGCTCGCACAGTTACTTGGCACTcactagttctagaGCggcc (SEQ ID NO: 61) |
| 2 | TTTTGTGATGCATGATTTGTTCTGTATTATCTTGCATTTCATTTTCATAAACTA |
| | ACTTGCCCCCGTTTTTATCCCTAGAAATTAGTACTTTTTTTCTATCAACCTCTA |
| | CTTTAGTAATTCTCATAGTTTTCACCTCAATGATTTTTTTCTCTCTTCTATTGT |
| | ACATATAATCACAAAAAAATAAAACACCTAAATGATGGATAAGCGTTTTTAT |
| | ACTTATCCAcatTAGACGTTTTAGTCCTCTTTCATATAGAGGTAGTCTCTTACG |
| | ATGATTAAAATGATGACTGAAAAACAAAAGTTTTAGTCCTCTTTCATATAGA |
| | GGTAGTCTCTTACTTTATTCGATAAAGACAGCACGAATAAAAAGTTTTAGTC |
| | CTCTTTCATATAGAGGTAGTCTCTTACAACAAATATGGAAAGTAATTTATTT |
| | AAATTGTTTTAGTCCTCTTTCATATAGAGGTAGTCTCTTACCCTACTTAATAA |
| | TAGTAATTAAAACAACCAATGTAAAGGATATAATCAATATATTTAAAGTTTG |
| | CACGAGAATGCAATCATTTTATTCATAAATATCATATCATTTATAAGCTCTAT |
| | TTTCCATTTTCTAAGGCTAATAAATAAAACTGCTGTACCTATGGATCTAAGG |
| | AAGACTTATGCACACAGTACAGCAACTTTTCAGCATGATTTGTGTTAAAAAC |
| | ATTTAATTTATTGTAGCAATTTCTTGAGAATCTATAAACCATTTTCCGGTTTC |
| | AATTTTAACCAACTCCAAATCTAACAATACTTGATTTTCGGTTTTCTTTTGAT |
| | TGTCGACTTGAAAAGCAGACCAGGCTCGCACAGTTACTTGGCACT (SEQ ID |
| | NO: 62) |
| 3 | ctcgtgctataattatactaattttACATCACGGAAAAAGGTTATGCTGCTTTTAAGACCCAC |
| | TTTCACATTTAAGTTGTTTTTCTAATCCGCATATGATCAATTCAAGGCCGAAT |
| | AAGAAGGCTGGCTCTGCACCTTGGTGATCAAATAATTCGATAGCTTGTCGTA |
| | ATAATGGCGGCATACTATCAGTAGTAGGTGTTTCCCTTTCTTCTTTAGCGACT |
| | TGATGCTCTTGATCTTCCAATACGCAACCTAAAGTAAAATGCCCCACAGCGC |
| | TGAGTGCATATAATGCATTCTCTAGTGAAAAACCTTGTTGGCATAAAAAGGC |
| | TAATTGATTTTCGAGAGTTTCATACTGTTTTTCTGTAGGCCGTGTACCTAAAT |
| | GTACTTTTGCTCCATCGCGATGACTTAGTAAAGCACATCTAAAACTTTTAGC |
| | GTTATTACGTAAAAAATCTTGCCAGCTTTCCCCTTCTAAAGGGCAAAAGTGA |
| | GTATGGTGCCTATCTAACATCTCAATGGCTAAGGCGTCGAGCAAAGCCCGCT |
| | TATTTTTTACATGCCAATACAATGTAGGCTGCTCTACACCTAGCTTCTGGGCG |
| | AGTTTACGGGTTGTTAAACCTTCGATTCCGACCTCATTAAGCAGCTCTAATG |
| | CGCTGTTAATCACTTTACTTTTATCTAATCTAGACATCATTAATTCCTCCTTTT |
| | TGTTGACATTATATCATTGATAGAGTTATTTGTCAAACTAGTTTTTTATTTGG |
| | ATCCCCTCGAGTTCATGAAAAACTAAAAAAAATATTGACACTCTATCATTGA |
| | TAGAGCATAATTAAAATAAGCTCTCTATCATTGATAGAGTGAGTTAAACCTA |
| | TAGGAGGAAAAAACGATGATCTACTATATAAAAGATTTAAAAGTGAAAGGA |
| | AAAATATTTGAAAATCTAATGAACAAAGAGGCTGTAGAAGGATTAATTACT |
| | TTTTTAAAGAAAGCGGAATTTGAGATATACTCAAGAGAAAATTATTCAAAAT |
| | ACAACAAATGGTTTGAAATGTGGAAAAGCCCAACTTCGAGCCTTGTGTTTTG |
| | GAAAAATTATAGTTTTCGCTGTCATCTTCTTTTTGTCATAGAAAAAGATGGTG |
| | AATGCCTTGGAATTCCTGCATCTGTTTTTGAATCTGTACTTCAAATTTACCTT |
| | GCGGATCCGTTCGCTCCCGATACGAAAGAACTTTTTGTTGAGGTTTGTAATTT |
| | ATATGAATGTTTAGCGGATGTCACTGTCGTAGAACATTTTGAAGCGGAAGAA |
| | TCAGCGTGGCATAAATTAACCCATAATGAGACCGAAGTATCAAAAAGAGTC |
| | TATAGTAAAGATGATGACGAACTTCTTAAATATATTCCAGAATTTCTTGACA |
| | CCATAGCGACAAACAAGAAAAGTCAAAAATACAATCAAATTCAAGGAAAA |
| | ATACAAGAAATTAATAAGGAAATAGCTACACTTTATGAATCGTCAGAGGAT |
| | TATATATTTACTGAATATGTTAGTAATTTATATAGAGAGTCTGCAAAGTTGG |
| | AGCAACACAGCAAACAAATTTTAAAAGAGGAGCTAAATGACTACAAGGATC |
| | ATGATGGTGATTATAAAGATCACGACATCGATTACAAAGATGATGACGATA |
| | AATAAGCctctcgcctgtcccctcagttcagtaatttc (SEQ ID NO: 63) |
| 4 | ctcgtgctataattatactaattttACATCACGGAAAAAGGTTATGCTGCTTTTAAGACCCAC |
| | TTTCACATTTAAGTTGTTTTTCTAATCCGCATATGATCAATTCAAGGCCGAAT |
| | AAGAAGGCTGGCTCTGCACCTTGGTGATCAAATAATTCGATAGCTTGTCGTA |
| | ATAATGGCGGCATACTATCAGTAGTAGGTGTTTCCCTTTCTTCTTTAGCGACT |
| | TGATGCTCTTGATCTTCCAATACGCAACCTAAAGTAAAATGCCCCACAGCGC |
| | TGAGTGCATATAATGCATTCTCTAGTGAAAAACCTTGTTGGCATAAAAAGGC |
| | TAATTGATTTTCGAGAGTTTCATACTGTTTTTCTGTAGGCCGTGTACCTAAAT |
| | GTACTTTTGCTCCATCGCGATGACTTAGTAAAGCACATCTAAAACTTTTAGC |
| | GTTATTACGTAAAAAATCTTGCCAGCTTTCCCCTTCTAAAGGGCAAAAGTGA |
| | GTATGGTGCCTATCTAACATCTCAATGGCTAAGGCGTCGAGCAAAGCCCGCT |
| | TATTTTTTACATGCCAATACAATGTAGGCTGCTCTACACCTAGCTTCTGGGCG |
| | AGTTTACGGGTTGTTAAACCTTCGATTCCGACCTCATTAAGCAGCTCTAATG |
| | CGCTGTTAATCACTTTACTTTTATCTAATCTAGACATCATTAATTCCTCCTTTT |
| | TGTTGACATTATATCATTGATAGAGTTATTTGTCAAACTAGTTTTTTATTTGG |
| | ATCCCCTCGAGTTCATGAAAAACTAAAAAAAATATTGACACTCTATCATTGA |
| | TAGAGCATAATTAAAATAAGCTCTCTATCATTGATAGAGTGAGTTAAACCTA |
| | TAGGAGGAAAAAACGATGATCTACTATATAAAAGATTTAAAAGTGAAAGGA |
| | AAAATATTTGAAAATCTAATGAACAAAGAGGCTGTAGAAGGATTAATTACT |
| | TTTTTAAAGAAAGCGGAATTTGAGATAGCCGCAAGAGAAGCATATTCAAAA |
| | TACAACAAATGGTTTGAAATGTGGAAAAGCCCAACTTCGAGCCTTGTGTTTT |
| | GGAAAAATTATAGTTTTCGCTGTCATCTTCTTTTTGTCATAGAAAAAGATGGT |
| | GAATGCCTTGGAATTCCTGCATCTGTTTTTGAAGCTGTACTTGCTATTTACCT |
| | TGCGGATCCGTTCGCTCCCGATACGAAAGAACTTTTTGTTGAGGTTTGTAATT |
| | TATATGAATGTTTAGCGGATGTCACTGTCGTAGAACATTTTGAAGCGGAAGA |
| | ATCAGCGTGGCATAAATTAACCCATAATGAGACCGAAGTATCAAAAAGAGT |
| | CTATAGTAAAGATGATGACGAACTTCTTAAATATATTCCAGAATTTCTTGAC |
| | ACCATAGCGACAAACAAGAAAAGTCAAAAATACAATCAAATTCAAGGAAA |
| | AATACAAGAAATTAATAAGGAAATAGCTACACTTTATGAATCGTCAGAGGA |
| | TTATATATTTACTGAATATGTTAGTAATTTATATAGAGAGTCTGCAAAGTTG |
| | GAGCAACACAGCAAACAAATTTTAAAAGAGGAGCTAAATGACTACAAGGAT |
| | CATGATGGTGATTATAAAGATCACGACATCGATTACAAAGATGATGACGAT |
| | AAATAAGCctctcgcctgtcccctcagttcagtaatttc (SEQ ID NO: 64) |
| 5 | ctcgtgctataattatactaattttACATCACGGAAAAAGGTTATGCTGCTTTTAAGACCCAC |
| | TTTCACATTTAAGTTGTTTTTCTAATCCGCATATGATCAATTCAAGGCCGAAT |
| | AAGAAGGCTGGCTCTGCACCTTGGTGATCAAATAATTCGATAGCTTGTCGTA |
| | ATAATGGCGGCATACTATCAGTAGTAGGTGTTTCCCTTTCTTCTTTAGCGACT |
| | TGATGCTCTTGATCTTCCAATACGCAACCTAAAGTAAAATGCCCCACAGCGC |
| | TGAGTGCATATAATGCATTCTCTAGTGAAAAACCTTGTTGGCATAAAAAGGC |
| | TAATTGATTTTCGAGAGTTTCATACTGTTTTTCTGTAGGCCGTGTACCTAAAT |
| | GTACTTTTGCTCCATCGCGATGACTTAGTAAAGCACATCTAAAACTTTTAGC |
| | GTTATTACGTAAAAAATCTTGCCAGCTTTCCCCTTCTAAAGGGCAAAAGTGA |
| | GTATGGTGCCTATCTAACATCTCAATGGCTAAGGCGTCGAGCAAAGCCCGCT |
| | TATTTTTTACATGCCAATACAATGTAGGCTGCTCTACACCTAGCTTCTGGGCG |
| | AGTTTACGGGTTGTTAAACCTTCGATTCCGACCTCATTAAGCAGCTCTAATG |
| | CGCTGTTAATCACTTTACTTTTATCTAATCTAGACATCATTAATTCCTCCTTTT |
| | TGTTGACATTATATCATTGATAGAGTTATTTGTCAAACTAGTTTTTTATTTGG |
| | ATCCCCTCGAGTTCATGAAAAACTAAAAAAAATATTGACACTCTATCATTGA |
| | TAGAGCATAATTAAAATAAGCTCTCTATCATTGATAGAGTGAGTTAAACCTA |
| | TAGGAGGAAAAAACGATGATCTACTATATAAAAGATTTAAAAGTGAAAGGA |
| | AAAATATTTGAAAATCTAATGAACAAAGAGGCTGTAGAAGGATTAATTACT |
| | TTTTTAAAGAAAGCGGAATTTGAGATATACTCAAGAGAAAATTATTCAAAAT |
| | ACAACAAATGGTTTGAAATGTGGAAAAGCCCAACTTCGAGCCTTGTGTTTTG |
| | GAAAAATTATAGTTTTCGCTGTCATCTTCTTTTTGTCATAGAAAAAGATGGTG |
| | AATGCCTTGGAATTCCTGCATCTGTTTTTGAATCTGTACTTCAAATTTACCTT |
| | GCGGATCCGTTCGCTCCCGATACGAAAGAACTTTTTGTTGAGGTTTGTAATTT |
| | ATATGAATGTTTAGCGGATGTCACTGTCGTAGAACATTTTTCAGCGTGGCAT |
| | AAATTAACCCATAATGAGACCGAAGTATCAAAAAGAGTCTATAGTAAAGAT |
| | GATGACGAACTTCTTAAATATATTCCAGAATTTCTTGACACCATAGCGACAA |
| | ACAAGAAAAGTCAAAAATACAATCAAATTCAAGGAAAAATACAAGAAATTA |
| | ATAAGGAAATAGCTACACTTTATGAATCGTCAGAGGATTATATATTTACTGA |
| | ATATGTTAGTAATTTATATAGAGAGTCTGCAAAGTTGGAGCAACACAGCAA |
| | ACAAATTTTAAAAGAGGAGCTAAATGACTACAAGGATCATGATGGTGATTA |
| | TAAAGATCACGACATCGATTACAAAGATGATGACGATAAATAAGCctctcgcctgt |
| | cccctcagttcagtaatttc (SEQ ID NO: 65) |
| 6 | ctcgtgctataattatactaattttACATCACGGAAAAAGGTTATGCTGCTTTTAAGACCCAC |
| | TTTCACATTTAAGTTGTTTTTCTAATCCGCATATGATCAATTCAAGGCCGAAT |
| | AAGAAGGCTGGCTCTGCACCTTGGTGATCAAATAATTCGATAGCTTGTCGTA |
| | ATAATGGCGGCATACTATCAGTAGTAGGTGTTTCCCTTTCTTCTTTAGCGACT |
| | TGATGCTCTTGATCTTCCAATACGCAACCTAAAGTAAAATGCCCCACAGCGC |
| | TGAGTGCATATAATGCATTCTCTAGTGAAAAACCTTGTTGGCATAAAAAGGC |
| | TAATTGATTTTCGAGAGTTTCATACTGTTTTTCTGTAGGCCGTGTACCTAAAT |
| | GTACTTTTGCTCCATCGCGATGACTTAGTAAAGCACATCTAAAACTTTTAGC |
| | GTTATTACGTAAAAAATCTTGCCAGCTTTCCCCTTCTAAAGGGCAAAAGTGA |
| | GTATGGTGCCTATCTAACATCTCAATGGCTAAGGCGTCGAGCAAAGCCCGCT |
| | TATTTTTTACATGCCAATACAATGTAGGCTGCTCTACACCTAGCTTCTGGGCG |
| | AGTTTACGGGTTGTTAAACCTTCGATTCCGACCTCATTAAGCAGCTCTAATG |
| | CGCTGTTAATCACTTTACTTTTATCTAATCTAGACATCATTAATTCCTCCTTTT |
| | TGTTGACATTATATCATTGATAGAGTTATTTGTCAAACTAGTTTTTTATTTGG |
| | ATCCCCTCGAGTTCATGAAAAACTAAAAAAAATATTGACACTCTATCATTGA |
| | TAGAGCATAATTAAAATAAGCTCTCTATCATTGATAGAGTGAGTTAAACCTA |
| | TAGGAGGAAAAAACGATGGCCTACGCTATAAAAGATTTAAAAGTGAAAGGA |
| | AAAATATTTGAAAATCTAATGAACAAAGAGGCTGTAGAAGGATTAATTACT |
| | TTTTTAAAGAAAGCGGAATTTGAGATATACTCAAGAGAAAATTATTCAAAAT |
| | ACAACAAATGGTTTGAAATGTGGAAAAGCCCAACTTCGAGCCTTGTGTTTTG |
| | GAAAAATTATAGTTTTCGCTGTCATCTTCTTTTTGTCATAGAAAAAGATGGTG |
| | AATGCCTTGGAATTCCTGCATCTGTTTTTGAATCTGTACTTCAAATTTACCTT |
| | GCGGATCCGTTCGCTCCCGATACGAAAGAACTTTTTGTTGAGGTTTGTAATTT |
| | ATATGAATGTTTAGCGGATGTCACTGTCGTAGAACATTTTGAAGCGGAAGAA |
| | TCAGCGTGGCATAAATTAACCCATAATGAGACCGAAGTATCAAAAAGAGTC |
| | TATAGTAAAGATGATGACGAACTTCTTAAATATATTCCAGAATTTCTTGACA |
| | CCATAGCGACAAACAAGAAAAGTCAAAAATACAATCAAATTCAAGGAAAA |
| | ATACAAGAAATTAATAAGGAAATAGCTACACTTTATGAATCGTCAGAGGAT |
| | TATATATTTACTGAATATGTTAGTAATTTATATAGAGAGTCTGCAAAGTTGG |
| | AGCAACACAGCAAACAAATTTTAAAAGAGGAGCTAAATGACTACAAGGATC |
| | ATGATGGTGATTATAAAGATCACGACATCGATTACAAAGATGATGACGATA |
| | AATAAGCctctcgcctgtcccctcagttcagtaatttc (SEQ ID NO: 66) |
| 7 | ctcgtgctataattatactaattttACATCACGGAAAAAGGTTATGCTGCTTTTAAGACCCAC |
| | TTTCACATTTAAGTTGTTTTTCTAATCCGCATATGATCAATTCAAGGCCGAAT |
| | AAGAAGGCTGGCTCTGCACCTTGGTGATCAAATAATTCGATAGCTTGTCGTA |
| | ATAATGGCGGCATACTATCAGTAGTAGGTGTTTCCCTTTCTTCTTTAGCGACT |
| | TGATGCTCTTGATCTTCCAATACGCAACCTAAAGTAAAATGCCCCACAGCGC |
| | TGAGTGCATATAATGCATTCTCTAGTGAAAAACCTTGTTGGCATAAAAAGGC |
| | TAATTGATTTTCGAGAGTTTCATACTGTTTTTCTGTAGGCCGTGTACCTAAAT |
| | GTACTTTTGCTCCATCGCGATGACTTAGTAAAGCACATCTAAAACTTTTAGC |
| | GTTATTACGTAAAAAATCTTGCCAGCTTTCCCCTTCTAAAGGGCAAAAGTGA |
| | GTATGGTGCCTATCTAACATCTCAATGGCTAAGGCGTCGAGCAAAGCCCGCT |
| | TATTTTTTACATGCCAATACAATGTAGGCTGCTCTACACCTAGCTTCTGGGCG |
| | AGTTTACGGGTTGTTAAACCTTCGATTCCGACCTCATTAAGCAGCTCTAATG |
| | CGCTGTTAATCACTTTACTTTTATCTAATCTAGACATCATTAATTCCTCCTTTT |
| | TGTTGACATTATATCATTGATAGAGTTATTTGTCAAACTAGTTTTTTATTTGG |
| | ATCCCCTCGAGTTCATGAAAAACTAAAAAAAATATTGACACTCTATCATTGA |
| | TAGAGCATAATTAAAATAAGCTCTCTATCATTGATAGAGTGAGTTAAACCTA |
| | TAGGAGGAAAAAACGATGATCTACTATATAAAAGATTTAAAAGTGAAAGGA |
| | AAAATATTTGAAAATCTAATGAACAAAGAGGCTGTAGAAGGATTAATTACT |
| | TTTTTAAAGAAAGCGGAATTTGAGATATACTCAAGAGAAAATTATTCAAAAT |
| | ACAACAAATGGTTTGAAATGTGGAAAAGCCCAACTTCGAGCCTTGTGTTTTG |
| | GAAAAATTACGCTGCTCGCTGTCATCTTCTTTTTGTCATAGAAAAAGATGGT |
| | GAATGCCTTGGAATTCCTGCATCTGTTTTTGAATCTGTACTTCAAATTTACCT |
| | TGCGGATCCGTTCGCTCCCGATACGAAAGAACTTTTTGTTGAGGTTTGTAATT |
| | TATATGAATGTTTAGCGGATGTCACTGTCGTAGAACATTTTGAAGCGGAAGA |
| | ATCAGCGTGGCATAAATTAACCCATAATGAGACCGAAGTATCAAAAAGAGT |
| | CTATAGTAAAGATGATGACGAACTTCTTAAATATATTCCAGAATTTCTTGAC |
| | ACCATAGCGACAAACAAGAAAAGTCAAAAATACAATCAAATTCAAGGAAA |
| | AATACAAGAAATTAATAAGGAAATAGCTACACTTTATGAATCGTCAGAGGA |
| | TTATATATTTACTGAATATGTTAGTAATTTATATAGAGAGTCTGCAAAGTTG |
| | GAGCAACACAGCAAACAAATTTTAAAAGAGGAGCTAAATGACTACAAGGAT |
| | CATGATGGTGATTATAAAGATCACGACATCGATTACAAAGATGATGACGAT |
| | AAATAAGCctctcgcctgtcccctcagttcagtaatttc (SEQ ID NO: 67) |
| 8 | ctcgtgctataattatactaattttACATCACGGAAAAAGGTTATGCTGCTTTTAAGACCCAC |
| | TTTCACATTTAAGTTGTTTTTCTAATCCGCATATGATCAATTCAAGGCCGAAT |
| | AAGAAGGCTGGCTCTGCACCTTGGTGATCAAATAATTCGATAGCTTGTCGTA |
| | ATAATGGCGGCATACTATCAGTAGTAGGTGTTTCCCTTTCTTCTTTAGCGACT |
| | TGATGCTCTTGATCTTCCAATACGCAACCTAAAGTAAAATGCCCCACAGCGC |
| | TGAGTGCATATAATGCATTCTCTAGTGAAAAACCTTGTTGGCATAAAAAGGC |
| | TAATTGATTTTCGAGAGTTTCATACTGTTTTTCTGTAGGCCGTGTACCTAAAT |
| | GTACTTTTGCTCCATCGCGATGACTTAGTAAAGCACATCTAAAACTTTTAGC |
| | GTTATTACGTAAAAAATCTTGCCAGCTTTCCCCTTCTAAAGGGCAAAAGTGA |
| | GTATGGTGCCTATCTAACATCTCAATGGCTAAGGCGTCGAGCAAAGCCCGCT |
| | TATTTTTTACATGCCAATACAATGTAGGCTGCTCTACACCTAGCTTCTGGGCG |
| | AGTTTACGGGTTGTTAAACCTTCGATTCCGACCTCATTAAGCAGCTCTAATG |
| | CGCTGTTAATCACTTTACTTTTATCTAATCTAGACATCATTAATTCCTCCTTTT |
| | TGTTGACATTATATCATTGATAGAGTTATTTGTCAAACTAGTTTTTTATTTGG |
| | ATCCCCTCGAGTTCATGAAAAACTAAAAAAAATATTGACACTCTATCATTGA |
| | TAGAGCATAATTAAAATAAGCTCTCTATCATTGATAGAGTGAGTTAAACCTA |
| | TAGGAGGAAAAAACGATGATCTACTATATAAAAGATTTAAAAGTGAAAGGA |
| | AAAATATTTGAAAATCTAATGAACAAAGAGGCTGTAGAAGGATTAATTACT |
| | TTTTTAAAGAAAGCGGAATTTGAGATATACTCAAGAGAAAATTATTCAAAAT |
| | ACAACAAATGGTTTGAAATGTGGAAAAGCCCAACTTCGAGCCTTGTGTTTTG |
| | GAAAAATTATAGTTTTCGCTGTCATCTTCTTTTTGTCATAGAAAAAGATGGTG |
| | AATGCCTTGGAATTCCTGCATCTGTTTTTGAATCTGTACTTCAAATTTACCTT |
| | GCGGATCCGTTCGCTCCCGATACGAAAGAACTTTTTGTTGAGGTTTGTAATTT |
| | ATATGAATGTTTAGCGGATGTCACTGTCGTAGAACATTTTGAAGCGGAAGAA |
| | TCAGCGTGGCATAAATTAACCCATAATGAGACCGAAGTATCAAAAAGAGTC |
| | TATAGTAAAGATGATGACGAACTTCTTAAATATATTCCAGAATTTCTTGACA |
| | CCATAGCGACAGACTACAAGGATCATGATGGTGATTATAAAGATCACGACA |
| | TCGATTACAAAGATGATGACGATAAATAAGCctctegcctgtcccctcagttcagtaatttc |
| | (SEQ ID NO: 68) |

TABLE S7
Synthetic RNA substrates used in this disclosure

| RNA | |
|---|---|
| oligo | Sequence |
| non- | GGCACACCCGCAGGGAGGAGCCAAAGCACGUCCAUCAUUC |
| target | CGUUGCCACAGCAGAAGCCC (SEQ ID NO: 69) |
| spc2 | GGCACACCCGCAGGGAAAUGUGAAAAUGCAGCGAGGGGUU |
| target | GACUAACACAGCAGAAGCCC (SEQ ID NO: 70) |

The following reference listing is not an indication that any of the references are material to patentability:

### REFERENCES

1. R. Barrangou et al., CRISPR provides acquired resistance against viruses in prokaryotes. Science 315, 1709-1712 (2007).
2. L. A. Marraffini, E. J. Sontheimer, CRISPR interference limits horizontal gene transfer in staphylococci by targeting DNA. Science 322, 1843-1845 (2008).
3. A. Bolotin, B. Quinquis, A. Sorokin, S. D. Ehrlich, Clustered regularly interspaced short palindrome repeats (CRISPRs) have spacers of extrachromosomal origin. Microbiology 151, 2551-2561 (2005).
4. F. J. Mojica, C. Diez-Villasenor, J. Garcia-Martinez, E. Soria, Intervening sequences of regularly spaced prokaryotic repeats derive from foreign genetic elements. J. Mol. Evol. 60, 174-182 (2005).
5. C. Pourcel, G. Salvignol, G. Vergnaud, CRISPR elements in Yersinia pestis acquire new repeats by preferential uptake of bacteriophage DNA, and provide additional tools for evolutionary studies. Microbiology 151, 653-663 (2005).
6. S. J. Brouns et al., Small CRISPR RNAs guide antiviral defense in prokaryotes. Science 321, 960-964 (2008).
7. O. O. Abudayyeh et al., C2c2 is a single-component programmable RNA-guided RNA-targeting CRISPR effector. Science 353, aaf5573 (2016).
8. G. Gasiunas, R. Barrangou, P. Horvath, V. Siksnys, Cas9-crRNA ribonucleoprotein complex mediates specific DNA cleavage for adaptive immunity in bacteria. Proc. Natl. Acad. Sci. U.S.A. 109, E2579-2586 (2012).
9. M. Jinek et al., A programmable dual-RNA-guided DNA endonuclease in adaptive bacterial immunity. Science 337, 816-821 (2012).
10. P. Samai et al., Co-transcriptional DNA and RNA Cleavage during Type III CRISPR-Cas Immunity. Cell 161, 1164-1174 (2015).
11. E. R. Westra et al., CRISPR immunity relies on the consecutive binding and degradation of negatively supercoiled invader DNA by Cascade and Cas3. Mol. Cell 46, 595-605 (2012).
12. K. S. Makarova et al., Evolutionary classification of CRISPR-Cas systems: a burst of class 2 and derived variants. Nat. Rev. Microbiol. 18, 67-83 (2020).
13. A. East-Seletsky et al., Two distinct RNase activities of CRISPR-C2c2 enable guide-RNA processing and RNA detection. Nature 538, 270-273 (2016).
14. A. J. Meeske, S. Nakandakari-Higa, L. A. Marraffini, Cas13-induced cellular dormancy prevents the rise of CRISPR-resistant bacteriophage. Nature 570, 241-245 (2019).
15. J. Bondy-Denomy, A. Pawluk, K. L. Maxwell, A. R. Davidson, Bacteriophage genes that inactivate the CRISPR/Cas bacterial immune system. Nature 493, 429-432 (2013).
16. B. J. Rauch et al., Inhibition of CRISPR-Cas9 with Bacteriophage Proteins. Cell 168, 150-158 ell( )(2017).
17. A. Pawluk et al., Naturally Occurring Off-Switches for CRISPR-Cas9. Cell 167, 1829-1838 e1829 (2016).
18. Y. Bhoobalan-Chitty, T. B. Johansen, N. Di Cianni, X. Peng, Inhibition of Type III CRISPR-Cas Immunity by an Archaeal Virus-Encoded Anti-CRISPR Protein. Cell 179, 448-458 e411 (2019).
19. N. D. Marino et al., Discovery of widespread type I and type V CRISPR-Cas inhibitors. Science 362, 240-242 (2018).
20. K. E. Watters, C. Fellmann, H. B. Bai, S. M. Ren, J. A. Doudna, Systematic discovery of natural CRISPR-Cas12a inhibitors. Science 362, 236-239 (2018).
21. A. L. Borges et al., Bacteriophage Cooperation Suppresses CRISPR-Cas3 and Cas9 Immunity. Cell 174, 917-925 e910 (2018).
22. M. Landsberger et al., Anti-CRISPR Phages Cooperate to Overcome CRISPR-Cas Immunity. Cell 174, 908-916 e912 (2018).
23. J. Rocourt, A. Schrettenbrunner, H. Hof, E. P. Espaze, [New species of the genus Listeria: Listeria seeligeri]. Pathol Biol (Paris) 35, 1075-1080 (1987).
24. L. Liu et al., The Molecular Architecture for RNA-Guided RNA Cleavage by Cas13a. Cell 170, 714-726 e710 (2017).
25. L. Liu et al., Two Distant Catalytic Sites Are Responsible for C2c2 RNase Activities. Cell 168, 121-134 e112 (2017).
26. I. M. Slaymaker et al., High-Resolution Structure of Cas13b and Biochemical Characterization of RNA Targeting and Cleavage. Cell Rep 26, 3741-3751 e3745 (2019).
27. B. Zhang et al., Structural insights into Cas13b-guided CRISPR RNA maturation and recognition. Cell Res. 28, 1198-1201 (2018).
28. C. Zhang et al., Structural Basis for the RNA-Guided Ribonuclease Activity of CRISPR-Cas13d. Cell 175, 212-223 e217 (2018).
29. A. P. Hynes et al., Widespread anti-CRISPR proteins in virulent bacteriophages inhibit a range of Cas9 proteins. Nat Commun 9, 2919 (2018).
30. J. Lee et al., Potent Cas9 Inhibition in Bacterial and Human Cells by AcrIIC4 and AcrIIC5 Anti-CRISPR Proteins. MBio 9, (2018).
31. O. O. Abudayyeh et al., RNA targeting with CRISPR-Cas13. Nature 550, 280-284 (2017).
32. D. B. T. Cox et al., RNA editing with CRISPR-Cas13. Science 358, 1019-1027 (2017).
33. R. Simon, U. Priefer, A. Puhler, A Broad Host Range Mobilization System for Invivo Genetic-Engineering—Transposon Mutagenesis in Gram-Negative Bacteria. Bio-Technology 1, 784-791 (1983).
34. P. Lauer, M. Y. Chow, M. J. Loessner, D. A. Portnoy, R. Calendar, Construction, characterization, and use of two Listeria monocytogenes site-specific phage integration vectors. J. Bacteriol. 184, 4177-4186 (2002).
35. A. J. Meeske, L. A. Marraffini, RNA Guide Complementarity Prevents Self-Targeting in Type VI CRISPR Systems. Mol. Cell 71, 791-801 e793 (2018).
36. G. Demarre et al., A new family of mobilizable suicide plasmids based on broad host range R388 plasmid (IncW) and RP4 plasmid (IncPalpha) conjugative machineries and their cognate Escherichia coli host strains. Res. Microbiol. 156, 245-255 (2005).
37. M. Fujita, Temporal and selective association of multiple sigma factors with RNA polymerase during sporulation in Bacillus subtilis. Genes Cells 5, 79-88 (2000).
38. S. Q. Zheng et al., MotionCor2: anisotropic correction of beam-induced motion for improved cryo-electron microscopy. Nat. Methods 14, 331-332 (2017).
39. A. Rohou, N. Grigorieff, CTFFIND4: Fast and accurate defocus estimation from electron micrographs. J Struct Biol 192, 216-221 (2015).
40. S. H. Scheres, RELION: implementation of a Bayesian approach to cryo-EM structure determination. J Struct Biol 180, 519-530 (2012).
41. A. Kucukelbir, F. J. Sigworth, H. D. Tagare, Quantifying the local resolution of cryo-EM density maps. Nat. Methods 11, 63-65 (2014).
42. P. Emsley, B. Lohkamp, W. G. Scott, K. Cowtan, Features and development of Coot. Acta Crystallogr D Biol Crystallogr 66, 486-501 (2010).
43. P. D. Adams et al., PHENIX: a comprehensive Python-based system for macromolecular structure solution. Acta Crystallogr D Biol Crystallogr 66, 213-221 (2010).
44. E. F. Pettersen et al., UCSF Chimera—a visualization system for exploratory research and analysis. J Comput Chem 25, 1605-1612 (2004).

While the disclosure has been particularly shown and described with reference to specific embodiments, it should be understood by those having skill in the art that various changes in form and detail may be made therein without departing from the spirit and scope of the present disclosure as disclosed herein.

1. An isolated or recombinantly expressed protein comprising the sequence of SEQ ID NO:1, or an amino acid sequence that is at least 90% identical to the sequence of SEQ ID NO:1, across a contiguous segment of SEQ ID NO:1 that is from 10-232 amino acids in length.

2. The protein of claim 1, wherein the protein comprises additional amino acids that are not part of SEQ ID NO:1, wherein optionally the additional amino acids comprise a purification tag or a nuclear localization signal.

3. The protein of claim 1, wherein the isolated protein comprises the sequence of SEQ ID NO:1.

4. The protein of claim 1, wherein the protein is present within a cell that is not Listeria seeligeri.

5. The protein of claim 4, wherein the protein is present in a prokaryotic or eukaryotic cell.

6. An expression vector encoding the protein of claim 1.

7. One or more cells comprising the expression vector of claim 6.

8. A method comprising expressing the protein of claim 1 in cells, and optionally separating the protein from the cells.

9. A method comprising introducing into one or more cells a protein of claim 1, or an expression vector encoding said protein, and wherein said protein is expressed by the expression vector if the expression vector is used, and wherein optionally expression of the protein from the expression vector is controlled by an inducible promoter.

10. The method of claim 9, wherein the expression vector is used, the method further comprising inducing expression of the protein from an inducible promoter that is operably linked to a sequence encoding the protein.

11. The method of claim 9, wherein the protein in the one or more cells inhibits Cas13a modification of RNA in the cells.

12. A method comprising introducing into cells a Cas13a protein or an expression vector encoding said Cas13a protein, wherein the Cas13a is targeted to an RNA of interest by a guide RNA, and wherein modification of the RNA of interest by Cas13a is inhibited or stopped by a protein of claim 1.

13. A pharmaceutical composition comprising the protein of claim 1.

14. A cDNA encoding the protein of claim 1.

15. A ribonucleoprotein comprising the protein of claim 1, wherein the ribonucleoprotein is present in a pharmaceutical composition, or in a cell that is not Listeria seeligeri.

16. A method comprising adding a protein of claim 1 to an assay, the assay comprising RNA from a biological sample, a Cas13, and a guide RNA targeted to an RNA polynucleotide that may be in the biological sample, and determining whether or not the Cas13 cleaves a reporter RNA that is added to the sample before or after addition of the protein.

17. The method of claim 16, wherein the RNA polynucleotide to which the guide RNA is present is in the sample, the method comprising detecting a detectable signal produced at least in part by Cas13 cleavage of the reporter RNA.

18. The method any claim 16, wherein the RNA polynucleotide to which the guide RNA is specific is present in the assay and comprises a viral mRNA, a viral genomic RNA, a viral subgenomic RNA, or a combination thereof.

19. The method of claim 18, wherein the assay is comprised by a container, or a lateral flow device.

20. The method of claim 17, comprising determining the presence of the viral RNA, the method further comprising administering to the individual from whom the biological sample was obtained an anti-viral agent, and/or one or more antibodies that bind with specificity to the virus.


SEQUENCE LISTING (70 sequences)

SEQ ID NO: 1 - protein, 232 aa, Listeria seeligeri
MIYYIKDLKVKGKIFENLMNKEAVEGLITFLKKAEFEIYSRENYSKYNKWFEMWKSPTSSLVFWKNYSFRCHLLFVIEKDGECLGIPASVFESVLQIYLADPFAPDTKELFVEVCNLYECLADVTVVEHFEAEESAWHKLTHNETEVSKRVYSKDDDELLKYIPEFLDTIATNKKSQKYNQIQGKIQEINKEIATLYESSEDYIFTEYVSNLYRESAKLEQHSKQILKEELN

SEQ ID NO: 2 - DNA, 7 bp, artificial sequence
  CRISPR sequence
gtttagt

SEQ ID NO: 3 - DNA, 20 bp, artificial sequence
  primer
ggcgtgaaaatcaacgaccc

SEQ ID NO: 4 - DNA, 20 bp, artificial sequence
  primer
tttgcttcaatgtcgccagc

SEQ ID NO: 5 - DNA, 45 bp, artificial sequence
  primer
gtaccgggccccccctcgaggttttgtgatgcatgatttgttctg

SEQ ID NO: 6 - DNA, 38 bp, artificial sequence
  primer
cggccgctctagaactagtgagtgccaagtaactgtgc

SEQ ID NO: 7 - DNA, 34 bp, artificial sequence
  primer
aaacttagtcaacccctcgctgcattttcacatt

SEQ ID NO: 8 - DNA, 34 bp, artificial sequence
  primer
ttacaatgtgaaaatgcagcgaggggttgactaa

SEQ ID NO: 9 - DNA, 34 bp, artificial sequence
  primer
ttacaaaaagaagctaaagaagtaaaagaagaag

SEQ ID NO: 10 - DNA, 34 bp, artificial sequence
  primer
aaaccttcttcttttacttctttagcttcttttt

SEQ ID NO: 11 - DNA, 43 bp, artificial sequence
  rimer
gtttaactcactctatcaatgatagagagcttattttaattat

SEQ ID NO: 12 - DNA, 40 bp, artificial sequence
  primer
gcaagacgtagcccagcgcgtcagtgccaagtaactgtgc

SEQ ID NO: 13 - DNA, 34 bp, artificial sequence
  primer
ttacggtgtagtcaaagtggagtttggaaatatg

SEQ ID NO: 14 - DNA, 19 bp, artificial sequence
  CCCAGCGAACCATTTGAGG
cccagcgaaccatttgagg

SEQ ID NO: 15 - DNA, 59 bp, artificial sequence
  primer
ttatgcatcccttaacttaaaacaattcatccagtaaaatataatattttattttctcc

SEQ ID NO: 16 - DNA, 44 bp, artificial sequence
  primer
tactggatgaattgttttaagttaagggatgcataaactgcatc

SEQ ID NO: 17 - DNA, 58 bp, artificial sequence
  primer
atccatggcctggatccatcaagcttaaaattagtataattatagcacgagctctgat

SEQ ID NO: 18 - DNA, 50 bp, artificial sequence
  primer
gcttgatggatccaggccatggatggcggccgcctctcgcctgtcccctc

SEQ ID NO: 19 - DNA, 42 bp, artificial sequence
  primer
acctcaaatggttcgctgggttaatcgcttgccctcatctgt

SEQ ID NO: 20 - DNA, 41 bp, artificial sequence
  primer
ctataattatactaattttacatcacggaaaaaggttatgc

SEQ ID NO: 21 - DNA, 68 bp, artificial sequence
  primer
taccgggccccccctcgaggttaatgatgatggtggtgatgcttcatcgttaatagcgttcttactag

SEQ ID NO: 22 - DNA, 61 bp, artificial sequence
  primer
cgtgctataattatactaattttatatattcgttgactacatttttctactataatagaag

SEQ ID NO: 23 - DNA, 53 bp, artificial sequence
  primer
tgaggggacaggcgagaggctattacattacagctagtgataagtatgtacag

SEQ ID NO: 24 - DNA, 63 bp, artificial sequence
  primer
cattgatagagtgagttaaacactttacaagtttaacatattatgttaatatataaatatagc

SEQ ID NO: 25 - DNA, 59 bp, artificial sequence
  primer
tgaggggacaggcgagaggcgcccatttattatttgttatatttgttgtaaaaatttac

SEQ ID NO: 26 - DNA, 47 bp, artificial sequence
  primer
attgatagagtgagttaaacctataggaggaaaaaacgatgatctac

SEQ ID NO: 27 - DNA, 51 bp, artificial sequence
  primer
gaggggacaggcgagaggcttaatttagctcctcttttaaaatttgtttgc

SEQ ID NO: 28 - DNA, 56 bp, artificial sequence
  primer
cattgatagagtgagttaaactaaaagaggagctaaattaaatgacaaatttaatc

SEQ ID NO: 29 - DNA, 59 bp, artificial sequence
  primer
gaggggacaggcgagaggcatttatataaaaagtttaaatttctgcattaaattcttgg

SEQ ID NO: 30 - DNA, 53 bp, artificial sequence
  primer
cattgatagagtgagttaaactttaggaggaattaaaatgaataaatttgcat

SEQ ID NO: 31 - DNA, 55 bp, artificial sequence
  primer
tgaggggacaggcgagaggcctgatgtattatattaatccttgctcttttttatc

SEQ ID NO: 32 - DNA, 34 bp, artificial sequence
  primer
aaaccaaggtaaatttgaagtacagattcaaaaa

SEQ ID NO: 33 - DNA, 34 bp, artificial sequence
  primer
aactttttgaatctgtacttcaaatttaccttgg

SEQ ID NO: 34 - DNA, 34 bp, artificial sequence
  primer
aaactcatttcttttgatttctaaaaatatagta

SEQ ID NO: 35 - DNA, 34 bp, artificial sequence
  primer
aactactatatttttagaaatcaaaagaaatgag

SEQ ID NO: 36 - DNA, 34 bp, artificial sequence
  primer
ttacaacaaatatggaaagtaatttatttaaatt

SEQ ID NO: 37 - DNA, 34 bp, artificial sequence
  primer
aaacaatttaaataaattactttccatatttgtt

SEQ ID NO: 38 - DNA, 34 bp, artificial sequence
  primer
ttactttattcgataaagacagcacgaataaaaa

SEQ ID NO: 39 - DNA, 34 bp, artificial sequence
  primer
aaactttttattcgtgctgtctttatcgaataaa

SEQ ID NO: 40 - DNA, 34 bp, artificial sequence
  primer
ttaccaactatcgaaattgattggaaaataaata

SEQ ID NO: 41 - DNA, 34 bp, artificial sequence
  primer
aaactatttattttccaatcaatttcgatagttg

SEQ ID NO: 42 - DNA, 50 bp, artificial sequence
  primer
ctcctcttttaaaatttgtttgtagatcatcgttttttcctcctatagtc

SEQ ID NO: 43 - DNA, 44 bp, artificial sequence
  primer
cgatgatctacaaacaaattttaaaagaggagctaaattaaatg

SEQ ID NO: 44 - DNA, 68 bp, artificial sequence
  primer
gcgaccacacccgtcctgtgttaatgatgatggtggtgatgcttcatcgttaatagcgttcttactag

SEQ ID NO: 45 - DNA, 34 bp, artificial sequence
  primer
ttacgatgattaaaatgatgactgaaaaacaaaa

SEQ ID NO: 46 - DNA, 34 bp, artificial sequence
  primer
aaacttttgtttttcagtcatcattttaatcatc

SEQ ID NO: 47 - DNA, 34 bp, artificial sequence
  primer
ttactacaaacaacacgtataaaaacaaaaaatt

SEQ ID NO: 48 - DNA, 34 bp, artificial sequence
  primer
aaacaattttttgtttttatacgtgttgtttgta

SEQ ID NO: 49 - DNA, 34 bp, artificial sequence
  primer
ttactaatagaagaggttgtaaaagattgtaaag

SEQ ID NO: 50 - DNA, 34 bp, artificial sequence
  primer
aaacctttacaatcttttacaacctcttctatta

SEQ ID NO: 51 - DNA, 34 bp, artificial sequence
  primer
ttactttattacaatatattccgcaaacattcaa

SEQ ID NO: 52 - DNA, 34 bp, artificial sequence
  primer
aaacttgaatgtttgcggaatatattgtaataaa

SEQ ID NO: 53 - DNA, 35 bp, artificial sequence
  primer
ttacttgcaaaaagaaattacaaaggactttcatt

SEQ ID NO: 54 - DNA, 35 bp, artificial sequence
  primer
aaacaatgaaagtcctttgtaatttctttttgcaa

SEQ ID NO: 55 - DNA, 34 bp, artificial sequence
  primer
ttacgaatctggacatttaattgattttgcaaaa

SEQ ID NO: 56 - DNA, 34 bp, artificial sequence
  primer
aaacttttgcaaaatcaattaaatgtccagattc

SEQ ID NO: 57 - DNA, 34 bp, artificial sequence
  primer
ttactaaagcaatagcgaaatacattgaagaaaa

SEQ ID NO: 58 - DNA, 34 bp, artificial sequence
  primer
aaacttttcttcaatgtatttcgctattgcttta

SEQ ID NO: 59 - DNA, 28 bp, artificial sequence
  primer
gtttgcctaaaaatgcgcttaaatcagc

SEQ ID NO: 60 - DNA, 26 bp, artificial sequence
  primer
cgtcgtgcaatgctaatcaagattgc

SEQ ID NO: 61 - DNA, 743 bp, artificial sequence
  synthetic gene fragment
ccgggccccccctcgaggttttgtgatgcatgatttgttctgtattatcttgcatttcattttcataaactaacttgcccccgtttttatccctagaaattagtactttttttctatcaacctctactttagtaattctcatagttttcacctcaatgatttttttctctcttctattgtacatataatcacaaaaaaataaaacacctaaatgatggataagcgtttttatacttatccacattagacgttttagtcctctttcatatagaggtagtctcttactgagaccagtctcggaagctcaaaggtctcagttttagtcctctttcatatagaggtagtctcttaccctacttaataatagtaattaaaacaaccaatgtaaaggatataatcaatatatttaaagtttgcacgagaatgcaatcattttattcataaatatcatatcatttataagctctattttccattttctaaggctaataaataaaactgctgtacctatggatctaaggaagacttatgcacacagtacagcaacttttcagcatgatttgtgttaaaaacatttaatttattgtagcaatttcttgagaatctataaaccattttccggtttcaattttaaccaactccaaatctaacaatacttgattttcggttttcttttgattgtcgacttgaaaagcagaccaggctcgcacagttacttggcactcactagttctagagcggcc

SEQ ID NO: 62 - DNA, 837 bp, artificial sequence
  synthetic gene fragment
ttttgtgatgcatgatttgttctgtattatcttgcatttcattttcataaactaacttgcccccgtttttatccctagaaattagtactttttttctatcaacctctactttagtaattctcatagttttcacctcaatgatttttttctctcttctattgtacatataatcacaaaaaaataaaacacctaaatgatggataagcgtttttatacttatccacattagacgttttagtcctctttcatatagaggtagtctcttacgatgattaaaatgatgactgaaaaacaaaagttttagtcctctttcatatagaggtagtctcttactttattcgataaagacagcacgaataaaaagttttagtcctctttcatatagaggtagtctcttacaacaaatatggaaagtaatttatttaaattgttttagtcctctttcatatagaggtagtctcttaccctacttaataatagtaattaaaacaaccaatgtaaaggatataatcaatatatttaaagtttgcacgagaatgcaatcattttattcataaatatcatatcatttataagctctattttccattttctaaggctaataaataaaactgctgtacctatggatctaaggaagacttatgcacacagtacagcaacttttcagcatgatttgtgttaaaaacatttaatttattgtagcaatttcttgagaatctataaaccattttccggtttcaattttaaccaactccaaatctaacaatacttgattttcggttttcttttgattgtcgacttgaaaagcagaccaggctcgcacagttacttggcact

SEQ ID NO: 63 - DNA, 1663 bp, artificial sequence
  synthetic gene fragment
ctcgtgctataattatactaattttacatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagcataattaaaataagctctctatcattgatagagtgagttaaacctataggaggaaaaaacgatgatctactatataaaagatttaaaagtgaaaggaaaaatatttgaaaatctaatgaacaaagaggctgtagaaggattaattacttttttaaagaaagcggaatttgagatatactcaagagaaaattattcaaaatacaacaaatggtttgaaatgtggaaaagcccaacttcgagccttgtgttttggaaaaattatagttttcgctgtcatcttctttttgtcatagaaaaagatggtgaatgccttggaattcctgcatctgtttttgaatctgtacttcaaatttaccttgcggatccgttcgctcccgatacgaaagaactttttgttgaggtttgtaatttatatgaatgtttagcggatgtcactgtcgtagaacattttgaagcggaagaatcagcgtggcataaattaacccataatgagaccgaagtatcaaaaagagtctatagtaaagatgatgacgaacttcttaaatatattccagaatttcttgacaccatagcgacaaacaagaaaagtcaaaaatacaatcaaattcaaggaaaaatacaagaaattaataaggaaatagctacactttatgaatcgtcagaggattatatatttactgaatatgttagtaatttatatagagagtctgcaaagttggagcaacacagcaaacaaattttaaaagaggagctaaatgactacaaggatcatgatggtgattataaagatcacgacatcgattacaaagatgatgacgataaataagcctctcgcctgtcccctcagttcagtaatttc

SEQ ID NO: 64 - DNA, 1663 bp, artificial sequence
  synthetic gene fragment
ctcgtgctataattatactaattttacatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagcataattaaaataagctctctatcattgatagagtgagttaaacctataggaggaaaaaacgatgatctactatataaaagatttaaaagtgaaaggaaaaatatttgaaaatctaatgaacaaagaggctgtagaaggattaattacttttttaaagaaagcggaatttgagatagccgcaagagaagcatattcaaaatacaacaaatggtttgaaatgtggaaaagcccaacttcgagccttgtgttttggaaaaattatagttttcgctgtcatcttctttttgtcatagaaaaagatggtgaatgccttggaattcctgcatctgtttttgaagctgtacttgctatttaccttgcggatccgttcgctcccgatacgaaagaactttttgttgaggtttgtaatttatatgaatgtttagcggatgtcactgtcgtagaacattttgaagcggaagaatcagcgtggcataaattaacccataatgagaccgaagtatcaaaaagagtctatagtaaagatgatgacgaacttcttaaatatattccagaatttcttgacaccatagcgacaaacaagaaaagtcaaaaatacaatcaaattcaaggaaaaatacaagaaattaataaggaaatagctacactttatgaatcgtcagaggattatatatttactgaatatgttagtaatttatatagagagtctgcaaagttggagcaacacagcaaacaaattttaaaagaggagctaaatgactacaaggatcatgatggtgattataaagatcacgacatcgattacaaagatgatgacgataaataagcctctcgcctgtcccctcagttcagtaatttc

SEQ ID NO: 65 - DNA, 1651 bp, artificial sequence
  synthetic gene fragment
ctcgtgctataattatactaattttacatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagcataattaaaataagctctctatcattgatagagtgagttaaacctataggaggaaaaaacgatgatctactatataaaagatttaaaagtgaaaggaaaaatatttgaaaatctaatgaacaaagaggctgtagaaggattaattacttttttaaagaaagcggaatttgagatatactcaagagaaaattattcaaaatacaacaaatggtttgaaatgtggaaaagcccaacttcgagccttgtgttttggaaaaattatagttttcgctgtcatcttctttttgtcatagaaaaagatggtgaatgccttggaattcctgcatctgtttttgaatctgtacttcaaatttaccttgcggatccgttcgctcccgatacgaaagaactttttgttgaggtttgtaatttatatgaatgtttagcggatgtcactgtcgtagaacatttttcagcgtggcataaattaacccataatgagaccgaagtatcaaaaagagtctatagtaaagatgatgacgaacttcttaaatatattccagaatttcttgacaccatagcgacaaacaagaaaagtcaaaaatacaatcaaattcaaggaaaaatacaagaaattaataaggaaatagctacactttatgaatcgtcagaggattatatatttactgaatatgttagtaatttatatagagagtctgcaaagttggagcaacacagcaaacaaattttaaaagaggagctaaatgactacaaggatcatgatggtgattataaagatcacgacatcgattacaaagatgatgacgataaataagcctctcgcctgtcccctcagttcagtaatttc

SEQ ID NO: 66 - DNA, 1663 bp, artificial sequence
  synthetic gene fragment
ctcgtgctataattatactaattttacatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagcataattaaaataagctctctatcattgatagagtgagttaaacctataggaggaaaaaacgatggcctacgctataaaagatttaaaagtgaaaggaaaaatatttgaaaatctaatgaacaaagaggctgtagaaggattaattacttttttaaagaaagcggaatttgagatatactcaagagaaaattattcaaaatacaacaaatggtttgaaatgtggaaaagcccaacttcgagccttgtgttttggaaaaattatagttttcgctgtcatcttctttttgtcatagaaaaagatggtgaatgccttggaattcctgcatctgtttttgaatctgtacttcaaatttaccttgcggatccgttcgctcccgatacgaaagaactttttgttgaggtttgtaatttatatgaatgtttagcggatgtcactgtcgtagaacattttgaagcggaagaatcagcgtggcataaattaacccataatgagaccgaagtatcaaaaagagtctatagtaaagatgatgacgaacttcttaaatatattccagaatttcttgacaccatagcgacaaacaagaaaagtcaaaaatacaatcaaattcaaggaaaaatacaagaaattaataaggaaatagctacactttatgaatcgtcagaggattatatatttactgaatatgttagtaatttatatagagagtctgcaaagttggagcaacacagcaaacaaattttaaaagaggagctaaatgactacaaggatcatgatggtgattataaagatcacgacatcgattacaaagatgatgacgataaataagcctctcgcctgtcccctcagttcagtaatttc

SEQ ID NO: 67 - DNA, 1663 bp, artificial sequence
  synthetic gene fragment
ctcgtgctataattatactaattttacatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagcataattaaaataagctctctatcattgatagagtgagttaaacctataggaggaaaaaacgatgatctactatataaaagatttaaaagtgaaaggaaaaatatttgaaaatctaatgaacaaagaggctgtagaaggattaattacttttttaaagaaagcggaatttgagatatactcaagagaaaattattcaaaatacaacaaatggtttgaaatgtggaaaagcccaacttcgagccttgtgttttggaaaaattacgctgctcgctgtcatcttctttttgtcatagaaaaagatggtgaatgccttggaattcctgcatctgtttttgaatctgtacttcaaatttaccttgcggatccgttcgctcccgatacgaaagaactttttgttgaggtttgtaatttatatgaatgtttagcggatgtcactgtcgtagaacattttgaagcggaagaatcagcgtggcataaattaacccataatgagaccgaagtatcaaaaagagtctatagtaaagatgatgacgaacttcttaaatatattccagaatttcttgacaccatagcgacaaacaagaaaagtcaaaaatacaatcaaattcaaggaaaaatacaagaaattaataaggaaatagctacactttatgaatcgtcagaggattatatatttactgaatatgttagtaatttatatagagagtctgcaaagttggagcaacacagcaaacaaattttaaaagaggagctaaatgactacaaggatcatgatggtgattataaagatcacgacatcgattacaaagatgatgacgataaataagcctctcgcctgtcccctcagttcagtaatttc

SEQ ID NO: 68 - DNA, 1483 bp, artificial sequence
  synthetic gene fragment
ctcgtgctataattatactaattttacatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagcataattaaaataagctctctatcattgatagagtgagttaaacctataggaggaaaaaacgatgatctactatataaaagatttaaaagtgaaaggaaaaatatttgaaaatctaatgaacaaagaggctgtagaaggattaattacttttttaaagaaagcggaatttgagatatactcaagagaaaattattcaaaatacaacaaatggtttgaaatgtggaaaagcccaacttcgagccttgtgttttggaaaaattatagttttcgctgtcatcttctttttgtcatagaaaaagatggtgaatgccttggaattcctgcatctgtttttgaatctgtacttcaaatttaccttgcggatccgttcgctcccgatacgaaagaactttttgttgaggtttgtaatttatatgaatgtttagcggatgtcactgtcgtagaacattttgaagcggaagaatcagcgtggcataaattaacccataatgagaccgaagtatcaaaaagagtctatagtaaagatgatgacgaacttcttaaatatattccagaatttcttgacaccatagcgacagactacaaggatcatgatggtgattataaagatcacgacatcgattacaaagatgatgacgataaataagcctctcgcctgtcccctcagttcagtaatttc

SEQ ID NO: 69 - RNA, 60 nt, artificial sequence
  RNA oligo
ggcacacccgcagggaggagccaaagcacguccaucauuccguugccacagcagaagccc

SEQ ID NO: 70 - RNA, 60 nt, artificial sequence
  RNA oligo
ggcacacccgcagggaaaugugaaaaugcagcgagggguugacuaacacagcagaagccc